(19) United States
(12) Patent Application Publication
(10) Pub. No.: US 2023/0015302 A1
(43) Pub. Date: Jan. 19, 2023

(54) METHODS FOR IDENTIFYING VIRAL INFECTIONS AND FOR ANALYZING EXOSOMES IN LIQUID SAMPLES BY RAMAN SPECTROSCOPY

(71) Applicant: microPhotonX GmbH, Tutzing (DE)

(72) Inventors: Karin Schütze, Tutzing (DE); Raimund Schütze, Tutzing (DE); Hesham Kamaleldin Aly Mahmoud Yosef, Penzberg (DE)

(73) Assignee: microPhotonX GmbH, Tutzing (DE)

(21) Appl. No.: 17/782,951

(22) PCT Filed: Dec. 4, 2020
(86) PCT No.: PCT/EP2020/084684

§ 371 Date: Jun. 6, 2022

(30) Foreign Application Priority Data
Dec. 6, 2019 (EP) 19214194.3
May 20, 2020 (EP) 20175814.1

(51) Int. Cl. G01N 21/65; G01N 33/50; B01L 3/00

(52) U.S. Cl. CPC G01N 21/65; G01N 33/5035; B01L 3/502753; B01L 3/502715; B01L 2300/0681

(57) ABSTRACT

The present invention relates to an in vitro method for analysing liquid samples as to the presence, identity and properties of a virus comprising: a) analyzing said liquid samples for a virus spectroscopically by means of spontaneous Raman spectroscopy; and b) comparing the spectroscopic data to a database and identifying said virus. The present invention further relates to an in vitro method for analyzing exosomes in a liquid sample of a subject comprising: a) isolating exosomes from the liquid sample; b) analyzing said exosomes spectroscopically by means of spontaneous Raman spectroscopy; and c) obtaining a Raman spectrum for said exosomes. The present invention also refers to a device for analysing a liquid sample as to the presence, identity and properties of viruses; and to a device for analyzing exosomes in a liquid sample. Also envisaged are a method for monitoring a viral infection in a cell or group of cells and a method of monitoring the antiviral treatment effect in a virus infected cell or group of cells, as well as a system comprising said device and a module comprising a database comprising reference values of Raman spectra.

### FIELD OF THE INVENTION

The present invention relates to an in vitro method for analysing liquid samples as to the presence, identity and properties of a virus comprising: a) analyzing said liquid samples for a virus spectroscopically by means of spontaneous Raman spectroscopy; and b) comparing the spectroscopic data to a database and identifying said virus. The present invention further relates to an in vitro method for analyzing exosomes in a liquid sample of a subject comprising: a) isolating exosomes from the liquid sample; b) analyzing said exosomes spectroscopically by means of spontaneous Raman spectroscopy; and c) obtaining a Raman spectrum for said exosomes. The present invention also refers to a device for analysing a liquid sample as to the presence, identity and properties of viruses; and to a device for analyzing exosomes in a liquid sample. Also envisaged are a method for monitoring a viral infection in a cell or group of cells and a method of monitoring the antiviral treatment effect in a virus infected cell or group of cells, as well as a system comprising said device and a module comprising a database comprising reference values of Raman spectra.

### BACKGROUND OF THE INVENTION

Viruses are sub-microscopic infectious agents that replicate only inside the living cells of an organism. Viruses can infect all types of life forms, from animals and plants to microorganisms, including bacteria. About 5000 virus species have been described so far. Upon infection, a host cell is forced to produce a huge number of copies of the original virus, i.e. a virus invades a host cell and proliferates using the metabolic system in the host cell. The process common to all virus infections is adsorption, entry into viral host cells, synthesis of viral constituents, assembly of viral constituents (formation of virus particles), and release of the virus out of the cell. Viruses display a wide diversity of shapes and morphologies. In general, viruses are much smaller than bacteria. Most viruses have a diameter between 20 and 300 nanometres.

When not inside an infected cell or in the process of infecting a cell, viruses exist in the form of virions, i.e. independent particles. These virions typically consist of: (i) the genetic material, i.e. molecules of DNA or RNA that encode the structure of the proteins by which the virus acts; (ii) a capsid which surrounds and protects the genetic material; and in some cases (iii) an outside envelope of lipids.

Viruses are responsible for some of the most disastrous pandemics of human history, including the smallpox pandemic, the Spanish flu of 1918 and recently the COVID-19 pandemic. In pandemics, a huge number of diagnostic tests has to be performed in a short period of time. The diagnostics should, in particular, be fast, cost-effective and suitably sensitive and specific.

The diagnostic detection of viruses has traditionally been based on the performance of viral cultures. This approach includes the inoculation of suitable cell lines such as canine kidney or rhesus monkey kidney cells with clinical samples and a subsequent propagation for 7 to 10 days to monitor the development of cytopathic effects. Other possibilities include the performance of ELISA-based tests. A further development in this respect is the provision of an europium based nanoparticle detection. The currently primarily used detection approach is based on nucleic acid analysis. These tests are mainly based on PCR techniques including reverse transcriptase steps. For example, the detection of influenza viruses is typically performed with a loop-mediated isothermal amplification-based assay (LAMP) (Zhang et al., J. Med. Virol., 2020, 92, 408-417). Also the use of DNA-microarrays and of sequence-based test are described (Vemula et al., 2016, Viruses, 8, 96). However, these approaches are usually expensive and tedious or, as in the case of antibody approaches, might require a longer time.

An alternative to the mentioned approaches is the use of a biosensor, which is considered faster and more sensitive (Younis et al., 2020, Nanosensors for Smart Cities, Elsevier, Chapter 19, 327-338). Yet, corresponding techniques are still based on ELISA and PCR steps.

Despite of the high success rate of these methods, they are generally time consuming and require extensive sample preparation. Further limitations include low specificity and sensitivity, the necessity of pre-cultivation of the viruses, requirement of labelling and high background noise, which delay or impact a reliable characterization of viruses. However, in particular during pandemic viral infections, a fast processing of a huge number of specimen is considered essential for a suitable health management strategy.

Extracellular vesicles (EVs) are a heterogeneous group of cell-derived membrane enclosed structures that are naturally released from a variety of cell types, including cancer cells. Exosomes, which are extracellular vesicles, are generated within the endosomal system as intraluminal vesicles and secreted during the fusion of multivesicular endosomes with the cell surface and have been identified as mediators of cell-to-cell communication by transferring bioactive molecules (e.g. nucleic acids, proteins and lipids) into recipient cells (van Niel et al., Nature Reviews Molecular Cell Biology 19, 213-228, 2018).

Currently, there is a growing interest in defining the clinical relevance of exosomes. Due to their presence and stability in most bodily fluids, exosomes have great potential to serve as a liquid biopsy tool in various diagnostic and therapeutic applications, for instance, as sources for biomarkers for disease detection and progression, since they contain molecules derived directly from the parent cell. In particular, cancer has been the subject of much investigation in exosome biology. It was reported that cancer derived exosomes facilitate tumor proliferation by altering the local tumor environment, and metastasis at distant organs. Hence, the characterization of exosomes and their cargo and surface proteins may allow earlier detection of diseases such as cancer and can improve prognosis and the rate of survival. Due to the ability of exosomes to cross the blood brain barrier, they can also serve as biomarkers for neurodegenerative disorders. For instance, it has been demonstrated that proteins characteristic of exosomes are accumulated in plaques of Alzheimer's patients, suggesting that exosomes play a role in the pathogenesis of Alzheimer's disease.

To date, several methods have been developed to isolate, detect and analyze exosomes. The most common protocol that is considered to be a gold standard for isolation of exosomes is differential centrifugation. This method, however, is very time and cost intensive, and results in low yield and low purity exosomes samples. For quantification of exosomes typically an enzyme-linked immunosorbent assay (ELISA) is used. This approach has, however, limitations as to the detection of exosomes with unknown surface markers and is generally rather time-consuming.

Some exosome based diagnostic approaches focus on one specific molecular component as a biomarker for the presence of diseased cells by elaborate genomic, proteomic, metabolomic and lipidomic studies. Examples are elevated levels of miR-210 in exosomes of leukemia patients, reduced expression of CD63 in exosomes of melanoma patients or the presence of flottilin 2 in exosomes of prostate cancer patients. However, despite the detailed molecular information provided by these techniques, they require complicated and time-consuming protocols. Furthermore, detection of subpopulations that are present at low frequencies is a rather challenging endeavor as these analyses are performed on the whole population of extracellular vesicles. As such, bulk analysis of low frequency components and changes therein represents a great difficulty.

In view of the above, there is a need for an improved in vitro analysis methodology, which allows for a rapid and reliable detection and evaluation of viruses in liquid samples or cultures of infected cells, as well as a need for an efficient, rapid and reliable approach for the isolation and molecular characterization of exosomes in a liquid sample.

### OBJECTS AND SUMMARY OF THE INVENTION

The present invention addresses these needs and provides in one main aspect an in vitro method for analysing liquid samples as to the presence, identity and properties of a virus comprising: a) analyzing said liquid samples for a virus spectroscopically by means of spontaneous Raman spectroscopy; and b) comparing the spectroscopic data to a database and identifying said virus. This approach is highly advantageous since obtaining and analysing liquid samples in vitro by means of spontaneous Raman spectroscopy is extremely fast, minimally invasive and much less costly to perform when compared to traditional PCR or ELISA procedures. In particular, the extremely time-consuming cultivation of viruses can largely be avoided, thus allowing for a direct diagnostic assessment within a short period of time well below 3 hours. In addition, it is easily accessible, may allow for stratification and real-time monitoring of therapies, and can easily be repeated. Furthermore, the methodology does not require the presence of specific target genes or proteins since it is based on the reaction of the liquid sample components on stimulation with laser radiation and the subsequent recording of spontaneous Raman spectra.

In one embodiment of said method, the presence of a virus is a virus infection of a cell and/or indicates a virus infected/affected cell.

In another embodiment, said liquid sample is a cell culture, whole blood, blood plasma, urine, lavage, smear, saliva or stool sample.

In yet another embodiment, said analyzing step a) comprises an examination of cells and/or cellular compartments and/or cellular components such as extracellular vesicles, comprised in said sample.

In a further embodiment, said examination comprises a separate examination of cellular compartments such as cell's cytoplasm and/or nucleus and/or nucleoli and/or mitochondria and/or lipid droplets.

In a preferred embodiment, said viruses or cells are either unaltered or have been fixated.

In another embodiment, the method additionally comprising as step a-(i) an isolation of the virus from the liquid sample.

According to a preferred embodiment, said step a-(i) is performed by cell lysis and subsequent centrifugation or filtration of said liquid sample, or by a centrifugation or filtration of said liquid sample or wherein the supernatant of a cell culture of infected cells is directly applied to a chip.

In yet another embodiment, said filtration is performed in a chip designed to size-exclude components within the liquid sample which are larger than the virus.

In a further embodiment, said viruses are enriched in a micro-chamber of the chip.

In a further embodiment, said chip is part of a microfluidic system.

According to a preferred embodiment, step a) comprises recording at least one Raman spectrum by means of Raman spectroscopy of a virus.

In a further embodiment, the analysis of step a) comprises collecting and arresting at least a group of viruses in an optical trap in order to record a Raman spectrum.

In one embodiment, the analysis of step a) comprises arresting a cell suspected to be virus infected/affected or a cell derived from a cell culture of infected cells in an optical trap in order to record a Raman spectrum. In a preferred embodiment, the step comprises collecting and arresting a group of free-floating viruses in an optical trap in order to record the Raman spectrum.

In another embodiment, said optical trapping forces are produced simultaneously by means of an excitation beam of a Raman spectroscopy system.

In another aspect, the present invention relates to a method for monitoring a viral infection in a cell or group of cells, preferably in a cell or group of cells in a cell culture.

In an embodiment of said method for monitoring the cell or group of cells is suspected to be infected, is derived from an infected cell or is a cell, which is deliberately infected with a virus.

In a further embodiment, said cell or group of cells is derived from a cell culture or a patient's sample.

In a preferred embodiment, said sample or cell culture derived cell or group of cells is or has been treated previous or during to the monitoring of the viral infection with an antiviral agent.

In a further preferred embodiment, said deliberate infection with a virus is performed at any time point or stage during and/or before the monitoring and/or may be repeated at least once.

In one embodiment of the method of monitoring, the method comprises recording at least one Raman spectrum by means of Raman spectroscopy of a virus in said cell or group of cells; or of a virus infected/affected cell or group of cells.

In a preferred embodiment, said recording is performed previous and/or subsequent to the viral infection.

In a further preferred embodiment said recording is performed 2, 3, 4, 5, 6, 7, 8, 9, 10 times or more often subsequent to the viral infection, preferably in fixed time intervals or according to a predetermined schedule.

A further aspect of the present invention relates to a method of monitoring the antiviral treatment effect in a virus infected/affected cell or group of cells.

In a preferred embodiment, the virus infected/affected cell or group of cells is a virus infected/affected cell or group of cells in a cell culture.

In one embodiment of the method of monitoring the antiviral treatment effect said antiviral treatment of a cell or group of cells is a treatment with an antiviral agent, or by analysing exosomes, preferably exosomes derived from liquid biopsies of a patient.

In a preferred embodiment, the antiviral agent is a natural substance such as a flavonoid or polyterpen, vitamin C, liquorice extract such as glycyrrhizin, desferal, or sorafenib.

In yet another embodiment, the method comprises recording at least one Raman spectrum by means of Raman spectroscopy of a virus in said cell or group of cells; or of a virus infected/affected cell or group of cells.

In a preferred embodiment, said recording is performed previous and/or subsequent to the antiviral treatment.

In a further embodiment, said recording is performed 2, 3, 4, 5, 6, 7, 8, 9, 10 times or more often subsequent to the antiviral treatment. It is particularly preferred to perform the recording in fixed time intervals or according to a predetermined schedule.

In a preferred set of embodiments of any of the monitoring methods as defined above, the growth and/or natural status of said cell or group of cells is controlled.

In a further preferred embodiment said control comprises control of temperature, oxygen, CO₂ and nutrient supply.

In another embodiment, said method additionally comprises introducing said cell or group of cells into a chip wherein said chip is part of a microfluidic system.

In yet another embodiment, the deliberate infection of a cell with a virus or said antiviral treatment is performed subsequent to the introduction of the cell or group of cells into the chip and/or after a control measurement.

In a further specific embodiment, said cells are floating in the chip due to microfluidic activities.

It is preferred that said cells are allowed to settle down in wells, preferably μ-wells, within the chip.

In yet another embodiment said deliberate infection of cells with a virus or said antiviral treatment is performed subsequent to the settling down of the cells into said well and/or after a control measurement.

In further embodiments, the method comprises recording at least one Raman spectrum in said chip and/or microfluidic system by means of Raman spectroscopy of a virus in said cell or group of cells; or of a virus infected/affected cell or group of cells.

It is particularly preferred that said recording of at least one Raman spectrum is performed periodically during the floating movement or when the cells are settled down in the wells.

In yet another preferred embodiment, the cell is collected and arrested in an optical trap in order to record the Raman spectrum.

In a further preferred embodiment, the cell is moved using focused lasers such as optical tweezers or UV-microbeams in order to transport the cell.

In a further particularly preferred embodiment, said optical trapping forces and/or transportation forces are produced simultaneously by means of an excitation beam of a Raman spectroscopy system and/or a separate laser.

In a further main aspect the present invention relates to an in vitro method for analyzing exosomes in a liquid sample of a subject comprising: (a) isolating exosomes from the liquid sample; (b) analyzing said exosomes spectroscopically by means of spontaneous Raman spectroscopy; and (c) obtaining a Raman spectrum for said exosomes. This method and a corresponding device according to the present invention offer a sensitive and low cost approach, which can be implemented for fast and non-invasive assessment of molecular identity, functionality and purity of exosomes. The claimed elements thus facilitate measuring, capturing and sorting of exosomes in solutions and thereby avoid lengthy molecular or chemical analysis steps, or time-consuming detection formats such as ELISA or Western blots. The methods and devices can advantageously be used in diagnostic setups, therapeutic monitoring approaches or for academic research. For example, in routine cancer studies the presently claimed elements provide several advantages, such as non-invasive access to obtaining samples, the assessment of disease response to different treatments, or the gathering of molecular details with prognostic implications.

In one embodiment, the isolation in step a) is performed on a chip designed to separate cells or cellular components from the liquid phase of the sample, wherein said separation is preferably performed via filtration, or immunocapture exosomes from the liquid sample on the chip.

In a further embodiment, the exosomes are enriched in a channel of the chip.

In another embodiment, said chip is a part of a microfluidic system.

In a preferred embodiment, the method additionally comprises as step d) a quantification of the isolated exosomes.

In one embodiment, the step of obtaining a Raman spectrum comprises recording at least one Raman spectrum by means of Raman spectroscopy of said liquid sample.

In a further embodiment, the method comprises the determination on the basis of the obtained Raman spectrum, whether said subject is affected by a disease.

In another embodiment, said determination comprises a comparison of the obtained Raman spectrum of step c) with a Raman spectrum obtained from the exosomes of a healthy subject or of a subject affected by a disease.

In yet another embodiment, said determination comprises a comparison of the obtained Raman spectrum of step c) with a reference spectrum, preferably derived from a database, thereby determining the identity of disease.

In a preferred embodiment, said liquid sample is a body fluid sample, preferably a plasma, blood, bile, urine, breast milk, saliva, pleural fluid, ascites, cerebrospinal fluid, amniotic fluid or bronchoalveolar lavage fluid sample.

In a further alternative embodiment, said exosomes are isolated by differential centrifugation, ultracentrifugation, density gradient centrifugation, extraction by using immunomagentic beads, chromatography, ultrafiltration separation, or membrane-mediated exosome separation.

In yet another embodiment, the method comprises conducting a statistical evaluation of the at least one Raman spectrum.

In a preferred embodiment, the method comprises a principal component analysis and/or a cluster analysis and/or linear discriminant analysis (LDA), wherein a predefined threshold value is used to differentiate between a liquid sample from a healthy and a diseased subject.

In a further embodiment, the evaluation of the Raman spectrum comprises a spectral analysis of the Raman spectrum.

In yet another embodiment, the evaluation of the Raman spectrum comprises collecting and arresting an exosome in an optical trap in order to record the Raman spectrum.

In a preferred embodiment, said optical trapping forces are produced simultaneously by means of an excitation beam of a Raman spectroscopy system.

In one embodiment, said liquid sample is provided in a microfluidic system or a microfluidic channel.

In another embodiment, said liquid sample is provided in an electrical gradient.

In yet another embodiment, automatic analysis comprises a scanning step, wherein Raman spectra are collected automatically in a defined area.

In a further aspect the present invention relates to an in vitro method for analysing whole blood samples or samples comprising cellular portions of blood as to their change due to the presence of a virus infection of erythrocytes present in the sample comprising: a) spectroscopically analyzing said samples for the status of hemoglobin or of constituents thereof by means of spontaneous Raman spectroscopy; and b) comparing the spectroscopic data to a database and detecting the effect of a virus infection.

In a specific embodiment modified hemoglobin or modified constituents of hemoglobin are indicative for the effect of a virus infection, preferably of a coronavirus conveyed infection.

In a further embodiment of said modification of hemoglobin or of constituents of hemoglobin is a change of porphyrin due to interaction with a virus protein.

In a particularly preferred embodiment said virus protein is a surface glycoprotein, preferably E2 glycoprotein, or a non-structural virus protein.

In yet another embodiment of any of the method as defined above, the method comprises conducting a statistical evaluation of at least one Raman spectrum.

In a preferred embodiment, the method comprises a principle component analysis and/or cluster analysis and/or a hierarchical cluster analysis and/or a linear discriminant analysis (LDA) of at least one Raman spectrum.

In a further preferred embodiment the method comprises a spectral analysis of the Raman spectrum.

In yet another preferred embodiment, the method comprises statistical evaluation and judgement on the basis of artificial intelligence and/or machine learning algorithms for complex matrix data evaluation.

In a preferred embodiment said method additionally comprises detection or registration of one or more further parameters including time point, temperature, measurement or activity identity, accessory information on the cell and/or virus.

In a particularly preferred embodiment said parameter is recorded in a database.

In a further embodiment, any method as defined above is performed computerbased, preferably automatically or semi-automatically.

In a further aspect the present invention relates to a device for analysing a liquid sample as to the presence, identity and properties of viruses, wherein the device comprises as a first unit (i) a chip, optionally comprising a filtering unit, as a second unit (ii) a Raman spectroscopy system; and as a third unit (iii) an evaluation module which is coupled to the Raman spectroscopy system.

In a preferred embodiment, said device comprises as fourth unit (iv) a microfluidic component for semi-automated measurements of viruses and/or for transporting viruses, cells, groups of virus or cells, or antiviral agents and/or for separating said liquid sample components or viruses or cells, which is coupled to the Raman spectroscopy system.

In another embodiment, said device further comprises a module allowing for cell culturing. It is particularly preferred that said module further allows for controlling growth and/or natural status of a cell or group of cells.

In yet another preferred embodiment said device further comprises a module for administering an antiviral agent to a cell or group of cells.

In a further preferred embodiment, said filtering unit of the chip is designed to size-exclude components within the liquid sample which are larger than viruses, thereby isolating said viruses.

It is particularly preferred that said evaluation module is configured to analyse an isolated cell or virus by comparing the Raman spectrum obtained from an isolated cell or virus with a reference spectrum, preferably derived from a database.

In a further aspect the present invention relates to a device for analyzing exocomes in a liquid sample, wherein the device comprises as a first unit (i) a chip comprising a filtering unit or immunocapturing unit capable of isolating exosomes from a liquid sample; as a second unit (ii) a Raman spectroscopy system with combined integrated simultaneous trapping features in order to record a Raman spectrum of a liquid sample; and as a third unit (iii) an evaluation module which is combined with the Raman spectroscopy system.

In one embodiment, said device comprises as a forth unit (iv) a microfluidic component for semi-automated measurement and/or transporting exosomes, cells, groups of exosomes or cells and/or separating said liquid sample components or exosomes which is coupled to the Raman spectroscopy system.

In a further embodiment, said filtering unit of the chip is designed to size exclude components within the liquid sample which are larger than exosomes, thereby isolating said exosomes.

In one embodiment, said immunocapturing unit of the chip is designed to capture exosomes via immunoaffinitive interactions between receptors on the surface of exosomes and ligands on the surface of the chip, thereby isolating said exosomes. In yet a further embodiment, the device is configured to identify a Raman spectrum of the exosomes associated with a disease of the subject.

In a specific embodiment of the device for analysing a liquid sample as to the presence, identity and properties of viruses or the device for analyzing exosomes as mentioned above, said device further comprises an integrated optical trapping module.

In another specific embodiment of the device for analysing a liquid sample as to the presence, identity and properties of viruses or the device for analyzing exosomes as mentioned above embodiment, said first unit and second unit is an integrated Raman trapping microscope-spectroscope system.

In another specific embodiment of the device for analysing a liquid sample as to the presence, identity and properties of viruses or the device for analyzing exosomes as mentioned above, said evaluation module is designed to perform principle component analysis and/or a normalization on specific band and/or a cluster analysis and/or a hierarchical cluster analysis and/or a LDA analysis and/or supervised cluster analysis and/or deep learning.

In a specific set of embodiments of the device for analysing a liquid sample as to the presence, identity and properties of viruses or the device for analyzing exosomes as mentioned above, the device is configured to perform any method as defined herein above.

In another embodiment, said disease as mentioned above is cancer, a neurodegenerative disease, diabetes mellitus or a viral infection.

In a further aspect the present invention relates to system comprising the device as defined above and a module comprising a database comprising reference values of Raman spectra obtained from a virus or a cell infected with a virus and/or from samples from healthy patients, and/or from samples from patients having cancer or a neurodegenerative disease, diabetes mellitus or a viral infection.

In yet another aspect the present invention relates to the use of a method as defined herein above, of the device as described as above, or of a system as described above for the detection of a virus or virus infection in a subject.

In another aspect the present invention relates to the use of the method, the device, or of the system as described above for the detection of a disease in a subject, preferably for the detection of cancer, a neurodegenerative disease, diabetes mellitus or a viral infection.

In a further preferred embodiment of the method, the device, the system or the use as defined above, said virus is a DNA or RNA virus.

In specific embodiments, said virus is dsDNA virus, preferably belonging to the order of Caudovirales, Herpesvirales or Ligamenvirales, or belongs to the family of Adenoviridae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae, Lavidaviridae, Marseilleviridae, Mimiviridae, Nimaviridae, Nudiviridae, Pandoraviridae, Papillomaviridae, Phycodnaviridae, Plasmaviridae, Polydnaviruses, Polyomaviridae, Poxviridae, Sphaerolipoviridae, Tectiviridae, Tristromaviridae or Turriviridae, such as a human papillomavirus (HPV), a herpes virus, or an adenovirus.

In further specific embodiments said virus is an ssDNA virus, preferably belonging to the family of Anelloviridae, Bacilladnaviridae, Bidnaviridae, Circoviridae, Geminiviridae, Genomoviridae, Inoviridae, Microviridae, Nanoviridae, Parvoviridae, Smacoviridae or Spiraviridae.

In further specific embodiments said virus is a dsDNA virus, preferably belonging to the family of Amalgaviridae, Birnaviridae, Chrysoviridae, Cystoviridae, Endornaviridae, Hypoviridae, Megabirnaviridae, Partitiviridae, Picobirnaviridae, Reoviridae, Totiviridae, Quadriviridae, Botybirnavirus, wherein said virus is more preferably a rotavirus.

In yet another group of embodiments, said virus is a negative strand ssRNA virus, preferably belonging to the order of Muvirales, Serpentovirales, Jingchuvirales, Mononegavirales, Goujianvirales, Bunyavirales, Articulavirales., or the family of Filoviridae, Paramyxoviridae, Pneumoviridae or Orthomyxoviridae, such as an RSV, metapneumovirus, or an influenza virus.

In a further group of embodiments, said virus is a positive strand ssRNA virus, preferably belonging to the order of Nidovirales, Picornavirales or Tymovirales, or to the family of Coronaviridae, Picornaviridae, Caliciviridae, Flaviviridae or Togaviridae, wherein said virus is more preferably a rhinovirus, Norwalk-Virus, Echo-Virus or enterovirus, or a Coronavirus or belongs to the group of Coronaviruses, or belongs to the group of alpha or beta coronaviruses, such as human or Microchiroptera (bat) coronavirus, most preferably a SARS-CoV-2 virus.

### BRIEF DESCRIPTION OF THE DRAWINGS

FIG. 1 shows a bright field image of Vero cells in culture as displayed within the computer screen and managed by the control software. Flags represent measurement points in the nucleus. For each Raman measurement the spectral data as well as the bright field image is stored together with microscope coordinates and settings.

FIG. 2 shows Raman spectra of SARS-CoV-2 infected Vero cells 6 hrs and 24 hrs after infection (nucleus measurements) compared with control cells having been exposed to identical culture conditions. Raman spectra measurements have been taken from nucleus area. Two time points were analyzed and compared with measurements of the corresponding control cells (cells having faced the same culture conditions yet without virus infection): First: 24 hrs after infection (30 infected and 30 control cells−1 spectrum from each cell) Second: 6 hrs after infection (30 infected and 10 control cells−10 spectra from each cell). Each line within the collected spectra represents one measurement.

FIG. 3 shows results of nucleus measurements PCA-score plots. The PCA-score plots indicate differences between control and SARS-CoV-2 infected Vero cells at 24 hrs and at 6 hrs incubation.

FIG. 4 shows results of nucleus measurements PCA-loadings. PCA loadings indicate differences between control and SARS-CoV-2 infected Vero cells at 24 hrs and at 6 hrs incubation. Clear changes in nucleic acids (DNA/RNA) profile of the nucleus are detectable (see arrows).

FIG. 5 shows Raman mean spectra of control vs SARS-CoV-2 infected Vero cells after 24 hrs incubation. Mean spectra depict an increase in RNA content (see wavenumber 813 cm⁻¹′) of Vero cells after 24 hrs of SARS-CoV-2 infection. Raman spectra were taken within the nucleus.

FIG. 6 shows Raman spectral classes of SARS-CoV-2 infected Vero cells after 24 hrs incubation. Cluster analysis of the Raman spectra reveals that 70% of cells are showing elevated RNA expression upon SARS-CoV-2 infection. Raman spectra were taken within the nucleus.

FIG. 7 shows Raman mean spectra of control vs Influenza Virus A (IVA) infected A549 cells. Raman mean spectra indicate changes in proteins and RNA of the cells upon IVA infection for 24 hr. All Raman spectra collected from infected cells are displaying these changes, indicating the impact induced by the IVA. Raman spectra were taken within the nucleus.

FIG. 8 shows a change of Raman spectra of A549 cells at different time points after IVA infection. Scores plot of the principal component analysis based on the Raman spectra of control vs IVA infected A549 cells collected at 3 time points of IVA infection (6, 16, and 24 hrs). PCA-scores indicate clear differences between control and IVA infected cells at 24 hrs and to a lesser extent at 16 and 6 hrs incubation. Raman spectra were taken within the nucleus.

FIG. 9 shows a bright-field image of supernatant of IVA producing A549 human lung cancer cell culture. The laser trapping collected a bulk of sub-micron particles (see circle).

FIG. 10 shows a MembraneSlide mounted onto a Raman-Microscope platform. Displayed is a ChannelSlide with two chambers. The bottom consists of borosilicate glass of about 0.17 mm thickness. The mesh size of the filter excludes particles of >1 μm and 2 μm, respectively. Other pore sizes are possible.

FIG. 11 depicts separation of particles by filtration. The sample is injected through the opening (channel entrance) of the chip (left) and pressed through the channel and membrane. Shown are typical bright-field images of a cell extract composed of cellular debris and submicron sized particles before filtration (A) and after filtration (B). Depending on the pore size the filter retains particles larger than 2 μm and 1 μm, respectively. Optical trapping enriches submicron sized particles (circle) for Raman spectra acquisition.

FIG. 12 shows Raman mean spectra of IVA, E. coli bacteria, and exosomes. After certain filtration steps samples from cell extracts are typically composed of bacteria, lipids, viruses and extracellular vesicles like exosomes. Raman spectra of IVA shows different Raman pattern than E. coli and exosomes, implying that the spectral band collected from a virus sample are basically from the IVA and not from bacteria or exosomes that can contaminate the sample.

FIG. 13 shows scores plot of the principal component analysis based on the Raman spectra of IVA, E. coli bacteria, and exosomes. Raman spectra collected from IVA were compared with typical E. coli bacteria and exosomes. Raman spectra of IVA shows different Raman pattern than E. coli and exosomes, implying that the spectral band collected from the virus sample are basically from the IVA and not from bacteria or exosomes that can contaminate the sample.

FIG. 14 shows a score plot of monkey kidney CV1 cells incubated with attenuated vaccinia virus (Lister strain). Raman spectra taken from cytoplasmic area show clear differences between incubated cells and control whilst Raman spectra taken from nuclear region did not reveal any difference.

FIG. 15 depicts Raman mean spectra of blood measurements from 6 COVID-19 patients and from 4 healthy donors. Clear differences are depicted at wavenumbers 555 cm⁻¹ and 1650 cm⁻¹ as well as at 1250 cm⁻¹ (see arrows).

FIG. 16 depicts score plots of blood measurements from 6 COVID-19 patients and from 4 healthy donors.

FIG. 17 shows a comparison of Raman results from whole blood of COVID-19 patients with erythrocyte blood products. The change of mean spectra between donors and COVID-19 patients (A, B) are comparable with those of erythrocyte blood product 42 days after donation (C, D). Main differences are at wavenumbers 555 cm′ as well as at 1642 cm⁻¹ and 1657 cm⁻¹, corresponding to changes in cysteine and amide I. Such changes correlate with change in structural conformation of the hemoglobin molecule.

FIG. 18 shows a BioRam® analysis workflow used for some embodiments of the present invention.

FIG. 19 shows a comparison of Raman results concerning exosomes from healthy subjects and from subjects afflicted by cancer. The figure provides mean spectra.

FIG. 20 shows a score plot after principal component analysis of the Raman results of exosomes from healthy subjects and from subjects afflicted by cancer.

FIG. 21 shows Raman spectra of exosomes extracted from patient's plasma. The exosome are either derived from subjects afflicted by a vascular disease or from subjects afflicted by colorectal cancer.

FIG. 22 shows a score plot after principal component analysis of the Raman results of exosomes extracted from patient's plasma as shown in FIG. 21. The exosome are either derived from subjects afflicted by a vascular disease or from subjects afflicted by colorectal cancer.

### DETAILED DESCRIPTION OF THE EMBODIMENTS

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use, there is no time or time interval coherence between the steps, i.e. the steps may be carried out simultaneously or there may be time intervals of seconds, minutes, hours, days, weeks, etc. between such steps, unless otherwise indicated.

It is to be understood that this invention is not limited to the particular methodology, protocols, reagents, etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one main aspect an in vitro method for analysing liquid samples as to the presence, identity and properties of a virus comprising: a) analyzing said liquid samples for a virus spectroscopically by means of spontaneous Raman spectroscopy; and b) comparing the spectroscopic data to a database and identifying said virus.

As used herein, the term “liquid sample” refers to a liquid material obtained via suitable methods from one or more biological organisms or comprising one or more biological organisms or subcellular particles such as viruses or processed after having been obtained. The liquid sample may further be material obtained from contexts or environments in which biological organisms or subcellular particles such as viruses are present, or processed variants thereof. Typically, the liquid sample is an aqueous sample. In preferred embodiments, it may comprise a bio-organic fluid obtained from the body of a mammal that is taken for analysis, testing, quality control, or investigation purposes. In a preferred embodiment, said liquid sample may be blood such as whole blood, blood components or banked blood, cellular portions of blood, bile, urine, saliva, nasal fluid, ear fluid sweat, sputum, semen, breast fluid, milk, colostrum, pleural fluid, ascites, cerebrospinal fluid, amniotic fluid or bronchoalveolar lavage fluid, gastric fluid, aqueous humor, vitreous humor, gastrointestinal fluid, exudate, transudate, pleural fluid, pericardial fluid, upper airway fluid, peritoneal fluid, liquid stool, fluid harvested from a site of an immune response, or fluid harvested from a pooled collection site. In further embodiments, the liquid sample may be cell culture sample, or be derived from a cell culture. In further embodiments, the sample may comprise free-floating viruses or virions. A “cell culture” as used in the context of the present invention relates to cells, preferably plant, animal or mammalian cells, more preferably human cells, which are grown under controlled conditions outside their natural environment. These conditions may vary for each cell type. They typically comprise the presence of a suitable vessel with a substrate or medium that supplies essential nutrients such as amino acids, carbohydrates, vitamins or minerals, as well as growth factors, hormones, and gases such as CO₂ and/or O₂. Furthermore, the physio-chemical environment including, for example, pH, osmotic pressure and temperature is typically controlled and can be adjusted in view of changes to the cell behaviour or fitness. Cells in a cell culture may be grown as adherent or monolayer culture and thus require a surface or an artificial substrate. Alternatively, the cells may be grown free floating in a suspension culture. Cells in the cell culture may be selected according to the specific test demands. The cells may comprise sample derived cells, or immortal cells which reproduce indefinitely if the optimal conditions are provided. For the determination of virus infection, preferably of coronavirus, e.g. SARS-CoV2 infection, VeroE6 cells, HAE cells, Huh7 cells, HRT18 cells. Particularly preferred is the use of genetically modified VeroE6 cells, e.g. VeroE6/TMPRSS2, which express large amounts of the transmembrane protease 2.

Furthermore, the liquid sample may contain a tissue extract derived from body tissues, e.g. tissues obtained via biopsy or resections, preferably from a eukaryotic organism, more preferably from a mammalian organism, even more preferably from a human being. The biopsy material may be derived, for example, from all suitable organs, e.g. the lung, the muscle, brain, liver, pancreas, stomach, heart, stomach, cardio-vascular system, heart, intestine etc., a nucleated cell sample, a fluid associated with a mucosal surface, or skin. The liquid sample may further be or contain a smear, mouth swab, or throat swab, or be derived therefrom, e.g. by dilution or lysis procedures in a suitable liquid or aqueous solution. In order to be extracted, the biopsy material or swab material is typically homogenized and/or lysed and/or resuspended in a suitable buffer solution as known to the skilled person. Such samples may, in specific embodiments, be preprocessed e.g. by enrichment steps and/or dilution steps and/or inhibition procedures etc.

In further embodiments, the liquid sample may comprise a plant cell extract or comprise plant cells. The cells may, for example, be derived from any type of plant, or plant cell cultures as known to the skilled person. The cells may further be processed or treated, e.g. separated from other cells, or specific components of the cells may be removed. The cells may be provided in the liquid sample as suspension, e.g. in a buffer. The buffer may comprise ingredients which are suitable for the viability of the plant cell including nutrients, inhibitors etc.

In specific embodiments, the “liquid sample” may also encompass a non-bioorganic fluid that is, for example, taken for analysis or quality control purposes, including but not limited to vaccines, liquid pharmaceutical formulations, medical solutions and drops, and the like.

In further specific embodiments, the “liquid sample” may encompass a fluid obtained from food, for example vegetables such as cabbage, salad, fruits, etc. The “liquid sample” may also be derived from drinks or drinkings in any form, water, beverages such as fruit juice, tea, coffee, milk, etc. The liquid sample may also be derived from solutions of medicinal products such as cell therapeutics, blood products, tissue grafts, etc., or from liquids obtained from medical devices such as scalpels, tubes, bottles, flasks, etc.

In further embodiments, the sample may be obtained from surfaces of medical instruments, door knobs, window handles, toilet seats, taps, computer keyboards, dishes etc. or any other surface or material which can be or has been touched by a person.

The term “virus” or “viruses” refers to a sub-cellular particle and a sub-microscopic infectious agent, that replicates only inside the living cells of an organism. Viruses can infect all types of life forms including animals, plants, as well as microorganisms such as bacteria and archaea. The term further includes smaller entities such as viroids or virusoides. The term, in particular, further includes virions, i.e. virus particles which are present outside of cells and may also be are considered as “free-floating viruses”. This term is independent of the origin of the virus, i.e. whether it has been released from a cell by a natural procedure, i.e. as part of the viral life cycle, or whether it has been released due to lysis or cell destruction of virus infected cells etc. A virion typically consist of: (i) the genetic material, i.e. molecules of DNA or RNA that encode the structure of the proteins by which the virus acts; (ii) a capsid which surrounds and protects the genetic material; and in some cases (iii) an outside envelope of lipids. The term “virus” refers to any virus (or virion) known to the skilled person. The term, in particular, relates to pathogenic viruses (virions) in the context of health and hygiene, or any other type of virus present in the environment of human beings. Further information can be derived, for example, from suitable database resources such as the Virus Pathogen Resource (ViPR) which is accessible at http://www.viprbrc.org; or NCBI Virus, which is accessible at http://www.ncbi.nlm.nih.gov/labs/virus/vssi/#/. In preferred embodiments, the virus is a virus associated with COVID-19 or similar diseases.

The “presence” of a virus refers to the physical presence of virus either outside of a cell or inside of cell. Should the virus be outside of a cell, i.e. be present as a virion as defined herein above, it may be analyzed in a liquid portion of the sample without further processing or modification, or, in alternative embodiments after certain separation, processing, drying etc. activities. Should the virus be inside of a cell, e.g. in situations where virus infected cells are analyzed, cell culture cells are analyzed, cells after separation from smaller entities such as viruses (virions) are analyzed or the like, the entire cell and/or sub-portions or compartments of a cell, e.g. the nucleus and/or the cytoplasm and/or the nucleolus and/or the mitochondria and/or lipid droplets etc. may be analyzed. The presence of a virus may accordingly be detected as virus infection of a cell or as indicative for a virus-infected cell. The method according to the present invention thus allows for the determination of a virus infection of a cell or cell type. The method according to the present invention further allows for the determination of virus infected cells, e.g. among other cells, which are not virus infected. A “virus affected cell” means a cell, which does not comprise a complete virus or a viral genome or is used by a virus for its replication but is directly or indirectly affected by a virus. Such an impact may be, for example, the introduction of viral proteins to the cytoplasm or nucleus of the cell, the detection of virus components by receptors, the activation of signalling cascades, the interaction of cells with neighbouring cells via exchange of factors or vesicles etc. The term “virus infected/affected cell” as used herein means that the cell may be virus infected or be virus affected, or, in specific embodiments, that the cell is virus infected and also affected as described above.

Further, the method according to the present invention allows for the determination of specific virus infections of cells, e.g. to distinguish between cells being infected by different viruses.

In specific embodiments, the method according to the present invention further envisages the analysis or examination of cellular components, e.g. in the form of extracellular vesicles. The term “cellular component” as used herein means that the component is of cellular origin, but not necessarily part of a cell. A cellular component may hence be a vesicle or other portion of a cell, irrespective of its presence in the intra- or extracellular space. The term “extracellular vesicle” as used herein relates to a liquid or cytoplasm enclosed by a lipid bilayer or membrane. Such a vesicle is typically cell derived but moves outside of a cell. Without wishing to be bound by theory, it is assumed that extracellular vesicles, e.g. exosomes, play a crucial role during viral infection processes. They may, for example, constitute crucial components in the pathogenesis of virus infection. They are further believed to produce effective immunity against virus infections by activating antiviral mechanisms and by transporting antiviral factors between adjacent cells. It is further assumed that extracellular vesicles may comprise viral components such as mRNA, miRNA, DNA, proteins, e.g. membrane-spanning proteins, enzymes, heat shock proteins, or immune-regulator molecules (see, for example, Crenshaw et al., 2018, The Open Virology Journal, 12, 134). The present invention thus envisages the analysis of extracellular vesicles with respect to the presence of viruses, viral components such as mRNA, miRNA, DNA, proteins, e.g. membrane-spanning proteins, enzymes, or heat shock proteins, as well as the presence of antiviral factors. According to the features of the Raman analysis as described herein, patterns of entities such as vesicles are detected. The patterns are based on the analysis of the sum of all molecules present. The methodology thus advantageously allows to distinguish between the mentioned entities, e.g. vesicles, on the basis of said pattern.

The “identity” of viruses refers to a characterization of the virus with respect to its taxonomic status. It is preferred that the identity of the virus also includes information on the pathologic status of the virus. The identity of the virus may be determined on the level of sub-species or variety, species, genus, family or order. For example, the affiliation of a virus to a specific species, a specific sub-species, a specific family or a specific order may be achieved when performing the present invention. In further specific embodiments, the determination of identity may include the differentiation of two or more virus sub-species, species, genus, family or orders when present in a liquid sample. The taxonomic basis for the characterization of the virus with respect to its identity may follow the skilled person's knowledge about current taxonomic definitions, e.g. fuelled by morphologic, biochemical or genetic properties of a virus. In specific embodiments, the identity of a virus may also be derived from the composition of an extracellular vesicle such as an exosome, e.g. an exosome comprising a certain type of combination of elements, e.g. DNA and proteins, or an exosome comprising a certain type of nucleic acid.

Non-limiting examples of viruses which can be identified according to the present invention include a DNA or RNA virus. The DNA virus may be a dsDNA virus. The dsDNA virus may belong, for example, to the order of Caudovirales, Herpesvirales or Ligamenvirales, or belongs to the family of Adenoviridae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae, Lavidaviridae, Marseilleviridae, Mimiviridae, Nimaviridae, Nudiviridae, Pandoraviridae, Papillomaviridae, Phycodnaviridae, Plasmaviridae, Polydnaviruses, Polyomaviridae, Poxviridae, Sphaerolipoviridae, Tectiviridae, Tristromaviridae or Turriviridae. Preferably, it is a human papillomavirus (HPV), a herpes virus, or an adenovirus.

The DNA virus may alternatively be an ssDNA virus. The ssDNA virus may, for example, belong to the family of Anelloviridae, Bacilladnaviridae, Bidnaviridae, Circoviridae, Geminiviridae, Genomoviridae, Inoviridae, Microviridae, Nanoviridae, Parvoviridae, Smacoviridae or Spiraviridae.

The RNA virus may be a dsRNA virus. The dsRNA virus may, for example, belong to the family of Amalgaviridae, Birnaviridae, Chrysoviridae, Cystoviridae, Endornaviridae, Hypoviridae, Megabirnaviridae, Partitiviridae, Picobirnaviridae, Reoviridae, Totiviridae, Quadriviridae, Botybirnavirus. Preferably, it is a rotavirus.

The RNA virus may alternatively be an ssRNA virus. For instance, said ssRNA virus may be a negative strand ssRNA virus. The negative strand ssRNA virus may, for example, belong to the order of Muvirales, Serpentovirales, Jingchuvirales, Mononegavirales, Goujianvirales, Bunyavirales or Articulavirales. Alternatively, the virus may be a negative strand ssRNA virus belonging to the family of Filoviridae, Paramyxoviridae, Pneumoviridae or Orthomyxoviridae. In a particularly preferred embodiment, the virus is an RSV, metapneumovirus, or an influenza virus.

The ssRNA may further be a positive strand ssRNA virus. Said positive strand ssRNA virus may, for example, belong to the order of Nidovirales, Picornavirales or Tymovirales. It is particularly preferred that said virus is a positive strand ssRNA virus belonging to the family of Coronaviridae, Picornaviridae, Caliciviridae, Flaviviridae or Togaviridae. It is further preferred that virus is a rhinovirus, Norwalk-Virus, Echo-Virus or enterovirus. It is even more preferred that the virus belongs to the family of Coronaviridae. Examples are Coronavirus or a member of the group of Coronaviruses. The group of Coronaviruses is typically divided into subgroups, i.e. alpha or beta coronaviruses. The present invention thus particularly envisages the identification or detection of a human coronavirus or a Microchiroptera (bat) coronavirus or a coronavirus obtained from a wild animal belonging to the group of pangolins or similar animals or belonging the Pholidota group.

In a particularly preferred embodiment said virus is PHEV, FcoV, IBV, HCoV-0C43 and HcoV HKU1, JHMV, HCoV NL63, HCoV 229E, TGEV, PEDV, FIPV, CCoV, MHV, BCoV, SARS-CoV, MERS-CoV or SARS-CoV-2, or any mutational derivative thereof. The term “mutational derivative thereof” as used herein relates to virus variants, which do not have the same genomic sequence as the mentioned viruses but are derived therefrom, e.g. by mutational events which are typical for this virus group. These events may lead, inter alia, to changes in the infectious behavior of the virus, but still allows for a classification of the virus, thus identification of the virus as belonging to the group of coronaviruses. It is most preferred that the virus is SARS-CoV-2.

In a further preferred embodiment, the virus to be detected or identified is a causative agent of a viral respiratory tract infection. Accordingly, the virus may belong to any of the above mentioned groups, families, classes or orders and be known to the skilled person as causing a viral respiratory tract infection. In a more preferred embodiment, the virus is a causative agent of MERS, SARS or COVID. In the most preferred embodiment, the virus is a causative agent of COVID-19, or a similar virally induced disease.

The term “properties of a virus” refers to an inherent or acquired characteristic of a virus. For example, the term may relate to a pathological status or quality of a virus, to a genetic or biochemical property, or to reactivity behaviour, preferably it relates to an infectiousness of a virus with respect to certain cell or cell type. The term “infectiousness” as used herein relates to the capacity of a virus to infect a certain cell, e.g. animal, preferably human cells. In specific embodiments, the infectiousness relates to the capacity of virus to bind to or attach to a certain cell, e.g. via a specific receptor at the surface of a cell and/or to become engulfed or incorporated by the cell, e.g. via endocytosis or membrane fusion. The cells which are infected by the virus show a corresponding infectability. The term “infectability” as used herein relates to the capacity of cells, e.g. animal, preferably human cells to become infected by a certain virus. In specific embodiments, the infectability relates to the capacity of cells to bind to or attach to a virus, e.g. via a specific receptor at the surface of a cell and/or to engulf or incorporate said virus, e.g. via endocytosis or membrane fusion.

The property of a virus may further refer to the sensitivity of viruses towards antiviral agents, e.g. compounds preventing entry of a virus into the cell, preventing energy consumption in a cell, preventing nucleic acid replication in a cell, preventing virus assembly in a cell, or preventing viral shedding. The term “antiviral agent” as used herein may include any suitable antiviral agent known to the skilled person, including a compound or agent, which is to be detected and described in the future. The term may, for example, refer to a class of medication used for treating viral infections. Such drugs may be designed to target specific viruses or a broad spectrum of viruses. Typical antiviral drugs include entry inhibitors, uncoating inhibitors, or inhibitors that target enzymes and processes during viral synthesis (e.g. reverse transcription/reverse transcriptase, integrase, transcription, translation, protease, protein processing and targeting, assembly, release). Examples of currently known antiviral agents which are envisaged by the present invention include Abacavir, Acyclovir, Adefovir, Amantadine, Ampligen, Amprenavir, Arbidol, Atazanavir, Atripla, Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, Ganciclovir, lbacitabine, lbalizumab, Idoxuridine, lmiquimod, Imunovir, Indinavir, Inosine, an integrase inhibitor, Interferons such as type I, type II, or type III Interferon, Lamivudine, Letermovir, Lopinavir, Loviride, Maraviroc, Methisazone, Moroxydine, Nelfinavir, Nevirapine, Nexavir, Nitazoxanide, Norvir, a nucleoside analogue, Oseltamivir, Peginterferon alfa-2a, Peginterferon alfa-2b, Penciclovir, Peramivir, Pleconaril, Podophyllotoxin, a protease inhibitor, Pyramidine, Raltegravir, Remdesivir, a reverse transcriptase inhibitor, Ribavirin, Rilpivirine, Rimantadine, Ritonavir, Saquinavir, Simeprevir, Sofosbuvir, Stavudine, a synergistic retroviral enhancer, Telaprevir, Telbivudine, Tenofovir alafenamide, Tenofovir disoproxil, Tenofovir, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine.

In preferred embodiments of the present invention the antiviral agent may further be a natural substance such as a flavonoid or polyterpen, vitamin C, liquorice extract such as glycyrrhizin, desferal, or sorafenib. The term “natural substance” refers to a compound that is found in nature and is produced by a living organism. However, this does not exclude the possible preparation of said natural substances by chemical synthesis. The term “flavonoid” relates to a class of polyphenolic plant pigments having a structure based on or similar to that of flavone, which fulfil functions in plants such as producing pigmentation in petals designed to attract pollinator animals, UV filtration, symbiotic nitrogen fixation, etc. Without wishing to be bound by theory it is assumed that flavonoids having protective properties against cancer and cardiovascular diseases, as well as antibacterial and in particular antiviral effects. Typical classes envisaged herein are anthocyanidins, anthoxanthins, flavanones, flavanonols, flavans and isoflavonoids, but are not limited thereto. A “polyterpen” is a natural or synthetic polymer of a terpene hydrocarbon. It may be produced by a variety of plants (e.g. confers), and by some insects. The term “liquorice extract” relates to a preparation that contains compounds of the flowering plant Glycyrrhiza glabra. The sweet-tasting constituent “glycyrrhizin” can be extracted from the plant. “Desferal” is a compound, which binds iron and aluminium and is also known as desferoxamine. Sorafenib (which is also known as Nexavar) is a kinase inhibitor drug used, which was established for the treatment of primary kidney cancer, advanced primary liver cancer, FLT3-ITD positive AML and advanced thyroid carcinoma. Also envisaged are combinations of the above mentioned antiviral agents, e.g. comprising any 2, 3, 4 or more of these agents.

In a first step of the method according to the present invention, the liquid sample as defined above is spectroscopically analysed for a virus by means of spontaneous Raman spectroscopy. The “spectroscopic analysis” as used herein generally relates to the analysis of viruses (virions) or exosomes present in a liquid sample or of cells comprising a virus by spectroscopic means, i.e. by studying the interaction of one or more viruses or exosomes or of cells or cellular compartments (comprising virus elements) and electromagnetic radiation. The analysis may further be performed in supernatants of suspensions comprising viruses or exosomes, e.g. after centrifugation as described herein below, or after separation of components in liquid samples via filtration steps as described herein below. The determination typically includes interaction with radiative energy as a function of its wavelength or frequency. By stimulating a virus or exosomes or cell and/or cellular compartments being influenced by a viral infection of a cell, an emission or response of the virus or exosome or the cell or cellular compartment is generated which can subsequently be recorded and analysed. The spectroscopy analysis which is to be performed according to the present invention is “Raman spectroscopy”. The method accordingly comprises recording or obtaining at least one Raman spectrum by means of Raman spectroscopy of a virus, a cell comprising a virus or being affected by a virus, or an exosome. The term “Raman spectroscopy” relates to a spectroscopic analysis which essentially relies on the observation of vibrational, rotational, and other low-frequency modes in a system. The technique is typically used to provide a structural fingerprint of molecules. It relies, in principle, on Raman scattering, i.e. inelastic scattering, of monochromatic light, from a laser in the visible, near infrared, or near ultraviolet range. The laser light typically interacts with molecular vibrations, phonons or other excitations in a system, e.g. a virus (virion) or exosome or a cell comprising a virus or being infected by a virus, resulting in the energy of the laser photons being shifted up or down. The shift in energy gives information about the vibrational modes in the system. Typically, a sample, e.g. comprising a virus or comprising a cell comprising a virus, or sub-portion thereof, e.g. a compartment of a cell such as the nucleus, a nucleolus, a mitochondrium, a lipid droplet or the cytoplasm, is illuminated with a laser beam. In preferred embodiments, the analysis comprises a separate or distinguished examination of a cell's cytoplasm and/or of a cell's nucleus any other compartment of a cell which can be suitably analyzed with the methods described herein. The compartment to the analysed may, in certain specific embodiments, be selected or changed according to the virus type and/or the time period after infection.

Electromagnetic radiation from the illuminated entity is collected with a lens and sent through a monochromator. Elastic scattered radiation at the wavelength corresponding to the laser light (i.e. Rayleigh scattering) may be filtered out, e.g. by a notch filter, an edge pass filter, or a band pass filter, while the rest of the collected light is dispersed onto a detector. In a typical embodiment, a Raman spectroscopy system may be used which comprises a light source which can in particular be a laser. The light source is typically configured to output an excitation beam. The excitation beam can for example have a wavelength in the range between 532 nm and 1064 nm, e.g. approximately 785 nm. Subsequently, a Raman spectrometer receives light scattered on the sample, e.g. a cell, by Stokes processes and/or Anti-Stokes processes. Furthermore, the approach may comprise the use of a Raman spectrometer comprising a diffractive element and an image sensor in order to record or obtain the Raman spectrum of the sample, e.g. a virus, an exosome, or a cell comprising a virus. Furthermore, additional elements may be employed to perform the analysis, e.g. focussing optical elements, which can be designed as lenses, and/or diaphragms. A “spontaneous Raman spectroscopy” means that the objects to be analyzed, e.g. viruses (virions), exosomes or cells comprising a virus or being infected by a virus, or the like, are unaltered, e.g. not previously prepared, lysed, processed, dried or otherwise modified in order to allow or facilitate the measurement. Instead, the spontaneous analysis is based on virus particles (virions) or exosomes in their native state or cells being infected by a virus in their native state, preferably in a liquid, e.g. aqueous environment. This approach allows for an extremely fast and artefact-free analysis, which is not possible if a set of sophisticated preparation steps has to be executed. In certain alternative embodiments, the viruses, exosomes, cells comprising a virus, or being infected with a virus, or any extracellular vesicle as defined herein may have been isolated, filtered, separated, purified, enriched or fixated before spectroscopic analysis. The present invention envisages any type of fixation, in particular chemical fixation or drying. It is preferred that a fixation provides a “freezing” effect on a cell and largely preserved the cell's molecular composition at the desired time of analysis. This is assumed to avoid changes to the Raman spectra due to cells dying outside optimal culture conditions, e.g. due to necrosis or apoptosis. Typically used fixatives include cell biology fixatives which preserve the metabolome. A preferred example is paraformaldehyde.

In contrast to SERS (surface enhanced Raman spectroscopy), spontaneous Raman spectroscopy as used in the context of the present invention refers to the detection of the spontaneous emission generated only by focused laser light, whereas SERS is based on stimulated emission which is generated by surface coating with various molecules or metallic substances. The SERS technique typically requires adsorption of the analyte molecules onto the SERS substrate. Upon adsorption onto the SERS surface, the Raman signal of the analyte is enhanced and the resultant signal intensity is comparable to that obtained by fluorescence. Unlike fluorescence, which exhibits broad adsorption/emission bands, the spectral peaks obtained in SERS are narrow. The high resolution of the SERS spectra makes simultaneous multicomponent analysis possible. Limitations of the SERS technique are (1) the method requires intimate contact between the enhancing surface and the analyte; (2) the substrates degrade with time resulting in a decrease in signal; (3) limited selectivity of the substrates for a given analyte; (4) limited re-usability of the substrates; and (5) problems with homogeneity and reproducibility of the SERS signal within a substrate (see also Mosier-Boss; Review of SERS Substrates for Chemical Sensing Nanomaterials 2017, 7, 142; doi:10.3390/nano7060142).

In addition, measuring single or multiple viruses (virions), exosomes or single cells comprising a virus or being infected by a virus is significantly improved due to optical trapping features, e.g. induced by focusing the Raman excitation laser through the objective of high numerical aperture. In a specific embodiment, an electromagnetic gradient may be induced. Thereby the viruses, exosomes or cells comprising a virus may be moved towards the central area of the focused laser beam and can be kept there during Raman spectrum acquisition. Further details may be derived from suitable literature sources such as Ashkin, 1970, Phys. Rev. Lett., 24, 156-159; or Ashkin & Dziedzic, 1987, Science, 235, 1517-1520.

The analysis of a virus, an exosome or of viruses or of cells comprising a virus or being infected by a virus by means of spontaneous Raman spectroscopy advantageously allows to draw conclusions on the identity and properties of a virus as defined herein.

In a further step of the method according to the present invention the susceptibility of isolated cells, e.g. virus infected/affected cells, preferably present in a suitable zone or micro-chamber of a chip as described herein, to an antiviral agent is determined by means of spontaneous Raman spectroscopy, i.e. the Raman spectroscopic technique, including a statistical evaluation, as described herein. The determination of susceptibility may preferably be performed at specific zones or areas of a chip as defined herein. For example, these zones or areas may comprise a predefined amount or concentration of a specific antiviral agent. The amount or concentration of the antiviral agent is typically based on the skilled person's knowledge of the antiviral agent's effect on virus infected/affected cells. For example, the concentration may be the MIC (minimal inhibitory concentration), i.e. the lowest concentration of a drug, which prevents reduce virus induced cytopathicity by 50%. Accordingly, the duration of antiviral agent exposure may, for example, be set in accordance with MIC parameters. It is preferred that the concentration of the antiviral agent is set to a value which is sufficiently high to indicate a reaction of the virus infected/affected cell to it. This value may be higher than the MIC, e.g. 10%, 25%, 50%, 75%, 100%, 200%, 500% etc. higher. The present invention envisages, in further specific embodiments, additional, different approaches, which make use of different concentrations and/or different exposure times, e.g. multiples of MIC. These parameters may further be adjusted during the performance of the method.

The measurement may be performed either with the same virus infected/affected cells which have before been analyzed via Raman spectroscopy after the supplementation with antiviral agents, e.g. via the microfluidic elements of the invention, or with different virus infected/affected cells.

The determination of antiviral agent susceptibility of virus infected/affected cells centrally comprises a comparison step of spontaneous Raman spectra obtained for a cell prior and subsequent to the exposure of the cell to the antiviral agent. “Prior to the exposure to the antiviral agent” refers to the acquirement of a Raman spectrum before the cells come into contact with an antiviral agent in specific areas within a micro-chamber the chip. There is no time restraint or limit as to the acquirement of such Raman spectra. The information may, in certain embodiments, have been obtained at any point of time in the past and also be derived from databases or previously recorded spectra or be additionally compared or supplemented with information from previously recorded spectra or database information. “Subsequent to the exposure to the antiviral agent” means obtaining a Raman spectrum after the cells have come into contact with an antiviral agent for a specific period of time, e.g. within a micro-chamber the chip.

In one embodiment, the cell may be exposed to the antiviral agent for about 0.5 to 30 minutes. Preferably the cell is exposed to the antiviral agent for about 0.5 to 5 minutes, about 5 to 10 minutes, about 10 to 15 minutes, about 15 to 20 minutes, about 20 to 25 minutes, or about 25 to 30 minutes. It is also envisaged to obtain Raman spectra at any other suitable intervals after the exposure to the antiviral agent. Preferably the Raman spectra are obtained at intervals of about one minute, about two minutes, about three minutes, about four minutes, about five minutes, about six minutes, about seven minutes, about eight minutes, about nine minutes, or about ten minutes. The intervals may further be combined with changes to the concentration of antiviral agent used, e.g. the concentration may be increased or decreased after one or more intervals, e.g. by 5%, 10%, 20%, 50%, 75% or 100%. In further preferred embodiments, the cells are exposed to one or more gradients of one or more antiviral agent. The gradients may be composed of different start and end concentrations and be provided within a micro-chamber as defined herein above, or along a tube or pathway being a part of the microfluidic system, or along a channel being part of the chip as defined herein. It is particularly preferred that the gradients are used with a group of cells, preferably of the same type or origin, which are located at different positions within the gradient, thus allowing for the determination of the working concentration of an antiviral agent. It is preferred to expose the cells according to the MIC value for the antiviral agent tested. It is also envisaged to obtain more than one Raman spectrum at the different intervals.

In certain embodiment, the virus infected/affected cell is exposed to a single antiviral agent, preferably to one of the antiviral agents mentioned above. In further embodiments, the cell is exposed to a combination of at least two different antiviral agents. The exposure may be performed simultaneously or sequentially. As used herein, “simultaneously” means a cell is exposed to a combination of at least two antiviral agents at the same time, by preferably using the MIC of the respective antiviral agent, whereas “sequentially” means a cell is exposed to a first antiviral agent followed by exposure to a second or further antiviral agent. In further embodiments the antiviral agent is provided to the cell at one or more micro-chambers within the chip. It is envisaged herein that one micro-chamber may contain one antiviral agent or a combination of at least two antiviral agents.

Upon exposure to the antiviral agent, the cell's physiology may be affected at many levels. For example, cells may respond to the antiviral agent by changing their morphology, macromolecular composition, metabolism, and/or gene expression. The changing morphology and physiology thus reflect the cell's susceptibility to the antiviral agent and can be determined by comparing the Raman spectrum prior and subsequently to exposure to an antiviral agent. This can typically be detected in a shifting, decrease or increase of peaks in the Raman spectrum, which are specific for a cell being infected with a virus in the context to the exposure to an antiviral agent, i.e. the cell's metabolic and infrastructure parameters change upon the effective exposure to an antiviral agent. The method of the present invention also envisages a kinetics study illustrating the sensitivity of cells to an antiviral agent or a combination of antiviral agents by recording Raman spectra at different intervals. In the case of virus infected/affected cells for which the antiviral agent does not work, no or slight changes in the Raman spectra are observed upon exposure to the antiviral agent or the combination of antiviral agent over time.

To facilitate the analysis of cells, exosomes or viruses and the determination of antiviral susceptibility of the cells or viruses as described above, a cell, virus or exosome may be transported or moved within the chip or microfluidic system, be collected, and/or arrested, e.g. in a micro-chamber, with the help of an optical trap. This further allows to suitably record a Raman spectrum of the trapped viruses, exosomes or cells. Accordingly, the present invention relates in a specific embodiment to the collection and arresting of a virus, more preferably of a group of viruses in an optical trap to record a Raman spectrum. In a further preferred embodiment the present invention relates to the collection and arrest of group of free-floating viruses in an optical trap in order to record the Raman spectrum. In an alternative embodiment, the present invention relater to the collection and arresting of an exosome, more preferably of a group of exosomes in an optical trap to record a Raman spectrum. The term “group” as used in the context of the arresting of the viruses, free-floating viruses or exosomes means a number of viruses or exosomes larger than about 5, preferably between about 5 and 500, e.g. 10, 20, 30, 40, 50, 60, 70, 80, 90, 100, 200, 300, 400, 450 or 500 particles. The term “optical trap” as used herein relates to a single-beam gradient force trap or optical tweezer, which uses a highly focused laser beam to provide an attractive or repulsive force. The optical trap may be produced by the excitation beam of the Raman spectroscopy system or a beam of electromagnetic radiation different therefrom. For example, a focal point of a beam may produce an optical trap potential, in which a cell is collected for the Raman spectroscopy. The focal point can be produced by the excitation beam, which is output by a light source. In such an embodiment, the excitation beam can thus be used both as excitation for the Raman scattering and for producing the optical trap. Alternatively, the optical trap can also be produced by a separate beam. The term “arrest” as used herein relates to a brief holding of a cell, virus or exosome or group of viruses or exosomes at a specific position to allow for the performance of Raman spectroscopy. Trapping forces can also be used to move the cells, viruses or exosomes within the channel or chamber of the chip or to transport them towards a microfluidic stream as mentioned herein. Alternatively, a pulse of the Raman excitation laser may be used. In specific embodiments, this pulse is used for catapulting or rapidly moving the cells, viruses or exosomes. Alternatively, a pulse from a UV laser (e.g. a 332 nm N-Laser) may be used. In further specific embodiments, the analysis of step a) as mentioned herein comprises arresting a cell infected by a virus, a cell affected by a virus, or a cell suspected to be virus infected, e.g. derivable from a sample obtained from a subject, in an optical trap in order to obtain a Raman spectrum. Alternatively, the cell arrested in the optical trap may be derived from a cell culture of infected cells as defined herein below.

In a preferred embodiment of the method as described above, the method comprises a comparison of the Raman spectrum obtained from the isolated virus, cell or exosome with a reference spectrum, thereby determining the identity of said virus. The term “reference spectrum”, as used herein, relates to a Raman spectrum obtained from a virus or exosome or virus infected/affected cell of known identity to be used as a matching template in order to designate a relation to a Raman spectrum obtained from a virus or exosome or virus of unknown identity or a cell, whose infection status is unknown, thereby identifying the unknown virus, or confirming that a cell is in fact virus-infected, preferably which virus is responsible for the infection of the cell. The spectrum may, for example, have been obtained previously or simultaneously from a control experiment. The control experiment may, for example, be performed with a predefined number of viruses, whose identity and/or properties are known, e.g. derived from biological material collection sites such as ATCC or DSMZ, or which have previously been determined and are cultivated, e.g. for control purposes or any other purpose. For example, control viruses, may be derived from the following groups: Caudovirales, Herpesvira les or Ligamenvirales, or belongs to the family of Adenoviridae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae, Lavidaviridae, Marseilleviridae, Mimiviridae, Nimaviridae, Nudiviridae, Pandoraviridae, Papillomaviridae, Phycodnaviridae, Plasmaviridae, Polydnaviruses, Polyomaviridae, Poxviridae, Sphaerolipoviridae, Tectiviridae, Tristromaviridae or Turriviridae, such as a human papillomavirus (HPV), a herpes virus, or an adenovirus, Anelloviridae, Bacilladnaviridae, Bidnaviridae, Circoviridae, Geminiviridae, Genomoviridae, Inoviridae, Microviridae, Nanoviridae, Parvoviridae, Smacoviridae, Spiraviridae, Amalgaviridae, Birnaviridae, Chrysoviridae, Cystoviridae, Endornaviridae, Hypoviridae, Megabirnaviridae, Partitiviridae, Picobirnaviridae, Reoviridae, Totiviridae, Quadriviridae, Botybirnavirus, rotavirus; Muvirales, Serpentovirales, Jingchuvirales, Mononegavirales, Goujianvirales, Bunyavirales, Articulavirales, Filoviridae, Paramyxoviridae, Pneumoviridae, Orthomyxoviridae, such as an RSV, metapneumovirus, or influenza virus, Nidovirales, Picornavirales, Tymovirales, Coronaviridae, Picornaviridae, Caliciviridae, Flaviviridae, Togaviridae, rhinovirus, Norwalk-Virus, Echo-Virus, enterovirus, alpha or beta coronaviruses, human or Microchiroptera (bat) coronavirus, SARS-CoV-2 virus.

In specific embodiments a reference or control spectrum may be obtained from cells in the cell culture, e.g. firstly a reference spectrum (or control) is obtained before infection with a virus. Subsequently, i.e. after infection, these cells are measured again, e.g. after a certain time. The measured cells may be the same cells measured before, yielding an individual cell kinetic, or the measurement is done with a cell population, e.g. the entire cell population. This yields a statistical overview of the change in the Raman spectra of the cell population over time. The obtained spectra can also be stored and used as reference spectra, for example to be able to allow a statement on the duration of an infection. Should there be uninfected cells, which may happen in some situations, a large group of cells may be measured and a cluster analysis is performed to determine how many subgroups are present in said group. On the basis of this information one may obtain spectra/information about the non-infected cells and store them, for example, as a reference.

In certain embodiments, one or more of the above mentioned viruses or any other suitable virus, or a cell infected with any of the above mentioned viruses may be provided in the chip, e.g. in one or more of the micro-chambers and be analysed together with the virus isolated from the liquid sample, the exosome isolated from the liquid sample, or the cell suspected to be virus infected/affected, or derived from a cell culture as described herein. Subsequently, a comparison of the obtained Raman spectra may be performed. The viruses or cells may be provided in the micro-chambers in a fixed form, e.g. via a PFA fixation. Further details would be known to the skilled person or can be derived from suitable literature sources such as Tabah et al., 2012, Intensive Care Med, 38, 1930-1945.

In a further step of the method according to the present invention, the spectroscopic data are compared to a database. The database comprises Raman spectra of reference particles or entities, e.g. of one or more viruses, or a reference spectrum as defined herein. In preferred embodiments, the database is an organized collection of Raman spectra obtained from a multitude of different virus species, e.g. those mentioned above, stored and accessed electronically from a computer system. The database specifically comprises reference spectra as described herein. The database may further comprise spectral information on previously measured spectra of control viruses, or of control samples, e.g. in the form of reference spectra, wherein the sample was exposed to one or more antiviral agents as mentioned herein. For instance, virus (virion) comprising samples could be measured by Raman spectroscopy to generate a Raman data library of defined native viruses (i.e. virions without interacting cells i.e. in solution). In a second step, unknown species can be measured and the resulting data compared with the data library to specify the species of the virus (virion) present in the sample. The present invention specifically envisages the generation of a database as mentioned above. This database may be provided with data derived from previous measurements and/or data from foreign sources such as literature sources or additional databases, e.g. from a network.

In specific embodiments, third party control samples or reference information may be used, e.g. derived from virus deposits, databases etc.

In preferred embodiments, the determination of spontaneous Raman spectroscopy comprises conducting a statistical evaluation of the at least one Raman spectrum, preferably of a plurality of Raman spectra, e.g. between 10 to 1000 spectra, by means of Raman spectroscopy of the virus or virus infected/affected cell or sub-portions thereof, e.g. compartments of a cell such as the nucleus and/or cytoplasm, or any other compartment of a cell which can be suitably analyzed with the spectroscopic methods described herein. It is particularly preferred to perform analyses in the nucleus compartment. The plurality of spectra may either be obtained for a single virus or cell, or for a group of viruses or cells, e.g. one spectrum may be obtained for one virus. It is particularly preferred to obtain spectra for single viruses or single virus infected/affected cells or single sub-portions thereof, e.g. compartments of a cell such as the nucleus or cytoplasm, e.g. via the use of optical traps as mentioned herein. It is further preferred that the statistical evaluation is a qualitative determination to which species, genus, family, order or group the virus or group of viruses belong to.

The statistical evaluation may, for example, be a principal component analysis (PCA) or a cluster analysis for each of the Raman spectra detected. Typically, in the “principal component analysis (PCA)”, a coordinate transformation in the N-dimensional data space is determined in such a way that the analysed entity of data points is spread along its most statistically relevant (e.g. variance-containing) coordinate axes in the transformed coordinate space. These coordinate axes define the principal components. The first principal component PC-1 typically defines the axis with the sharpest differences between the different groups of Raman spectra. “Cluster analysis” relates to a technique to group similar observations into a number of clusters based on the observed values of several variables for each individual. Cluster analysis maximizes the similarity of cases within each cluster while maximizing the dissimilarity between groups that are initially unknown.

Accordingly, by means of a statistical analysis such as the principal component analysis or a cluster analysis, as mentioned herein, it can be determined whether the pattern of Raman peaks contained in the Raman spectrum is characteristic of a specific virus or virus infected/affected cell, or of an exosome, e.g. a specific exosome (comprising a unique combination of surface factors and/or ingredients). Without wishing to be bound by theory, it is assumed that different viruses initiate different cascades of metabolic reactions, which can be detected via Raman spectroscopy. Alternatively or additionally, it can be determined whether the pattern of Raman peaks contained in the Raman spectrum is characteristic of the presence of a virus or of a virus infected/affected cell or of an exosome, or of a sub-portion of a virus infected/affected cell such as the nucleus or cytoplasm as a “photonic fingerprint”. A principal component analysis may hence be performed for a Raman spectrum or a plurality of Raman spectra which have been recorded from the sample, e.g. a virus, exosome or group of viruses, or a virus infected/affected cell or of a sub-portion of a virus infected/affected cell such as the nucleus or cytoplasm.

“Linear discriminant analysis (LDA)” or “normal discriminant analysis” or “discriminant function analysis” a dimensionality reduction technique which is commonly used in the pre-processing step for pattern-classification and machine learning applications.

The aim of this approach is to project a dataset onto a lower-dimensional space with good class-separability in order to avoid overfitting.

The term “spectral analysis” also refers to the evaluation of characteristic spectral patterns. As such, the determination of whether the Raman spectrum is characteristic of a virus or virus infected/affected cell, or exosome may hence not be based on individual Raman peaks, but rather on a plurality of Raman intensities distributed evenly or unevenly over the Raman spectra at a plurality of Raman wavenumbers, yielding a characteristic spectral pattern. Thus, by means of a statistical method such as the principal component analysis, as mentioned above, or other statistical methods such as cluster analyses, one can take advantage of the fact that the Raman spectrum as a whole shows characteristics that are indicative and specific of a virus, of an exosome, of a particular species of a virus or exosome, or of a cell infected with a virus of a particular species.

The pattern in a Raman spectrum can be defined by one or a plurality of parameters selected from the group composed of the wavenumbers at which the Raman peaks are located, the peak heights, the flank steepness of the peaks, the distances between the peaks, and/or combinations of peaks in one or a plurality of Raman spectra. For evaluation of one or a plurality of Raman spectra detected, e.g. for one virus or virus infected/affected cell, or one exosome, one can determine whether these peak(s) are situated in a space, according to a principal component analysis, in an area assigned to viruses, exosomes or virus infected/affected cells or in another area assigned to different entities, e.g. cells which have not been infected/affected by a virus.

For example, by means of a statistical evaluation, each Raman spectrum can be assigned to a point in an N-dimensional data space, wherein N>>1, e.g. N>100. The N-dimensional data space can be the data space spanned in a principal component analysis by the various principal components. Advantageously, one can determine from reference spectra, e.g. control experiments or previously recorded spectra, preferably as defined above, in which areas of the N-dimensional data space Raman spectra are arranged in clusters for viruses, exosomes or virus infected/affected cells and in which other areas of the N-dimensional data space Raman spectra are arranged in clusters for other entities, e.g. cells which have not been infected by a virus.

An assignment to the species of viruses or exosomes can further take place for a cluster analysis or for a different analysis of the recorded Raman spectra for example by means of different wavenumber ranges. For instance, in order to identify SARS-CoV-2, at least one, 2, 3, 4, 5 or 6 wavenumber(s) of 717 cm⁻¹, 813 cm⁻¹, 936 cm⁻¹, 1005 cm⁻¹, 1066 cm⁻¹, 1087 cm⁻¹, 1110 cm⁻¹, 1160 cm⁻¹, 1176 cm⁻¹, 1252 cm⁻¹, 1448 cm⁻¹, 1525 cm⁻¹ and 1656 cm⁻¹ may be detected. In addition, at least one, 2, 3, 4, 5 or 6 wavenumber(s) from one or a plurality of wavenumber ranges of 1650 to 1600 cm⁻¹, from 1350 to 1250 cm⁻¹, from 1180 cm⁻¹ to 1120 cm⁻¹, from 1100 cm⁻¹ to 1050 cm⁻¹, from 930 cm⁻¹ to 890 cm⁻¹ or from 700 cm⁻¹ to 650 cm⁻¹ may be evaluated. In order to perform the cluster analysis, the mentioned wavenumber ranges do not necessarily have to be evaluated, but rather other principal components can also be evaluated.

In a further preferred embodiment, the method comprises a statistical evaluation and judgment on the basis of artificial intelligence and/or machine learning algorithms for complex matrix data evaluation. The term “artificial intelligence” as used herein generally refers to supervised learning approaches. The term includes, inter alia, machine learning concepts. “Machine learning” as used herein typically relies on a two-step approach: first, a training phase; and second, a prediction phase. In the training phase, values of one or more parameters of the machine-learning model (MLM) are set using training techniques and training data. In the prediction phase, the trained MLM operates on measurement data. Example parameters of an MLM include: weights of neurons in a given layer of an artificial neural network (ANN) such as a convolutional neural network (CNN); kernel values of a kernel of a classifier etc. Building an MLM can include the training phase to determine the values of the parameters. Building an MLM can generally also include determining values of one or more hyperparameters. Typically, the values of one or more hyperparameters of the MLM are set and not altered during the training phase. Hence, the value of the hyperparameter can be altered in outer-loop iterations; while the value of the parameter of the MLM can be altered in inner-loop iterations. Sometimes, there can be multiple training phases, so that multiple values of the one or more hyperparameters can be tested or even optimized. The performance and accuracy of most MLMs are strongly dependent on the values of the hyperparameters. Exemplary hyperparameters include: number of layers in a convolutional neural network; kernel size of a classifier kernel; input neurons of an ANN; output neurons of an ANN; number of neurons per layer; learning rate; etc. Various types and kinds of MLMs can be employed in the context of the present invention. For example, a novelty detector MLM/anomaly detector MLM, or a classifier MLM may be employed, e.g., a binary classifier. For example, a deep-learning (DL) MLM can be employed: here, features detected by the DL MLM may not be predefined, but rather may be set by the values of respective parameters of the model that can be learned during. As a general rule, various techniques can be employed for building the MLM. For example, typically, the type of the training can vary with the type of the MLM. Since the type of the MLMs can vary in different implementations, likewise, the type of the employed training can vary in different implementations. For example, an iterative optimization could be used that uses an optimization function that is defined with respect to one or more error signals. For example, a backpropagation algorithm can be employed. The artificial intelligence and/or machine learning algorithms may, for example, advantageously be used to differentia between virus types or virus species, or between exosomes or exome types.

In a preferred set of embodiments, the in vitro method for analyzing liquid samples as to the presence, identity and properties of a virus according to the present invention additionally comprises as step a-(i) an isolation step of a virus from the liquid sample. As used herein, the term “isolation” or “isolating” refers to a process of removing or otherwise setting apart or separating viruses from their original liquid sample and/or from other components in said liquid sample. The term may further relate to a process of concentration of viruses within the original liquid sample, whereby significant amounts of the original liquid sample are removed, while viruses are not removed. The term may, in certain embodiments, further include an at least partial purification of viruses from the liquid sample, or from any non-virus or non-viral component within the sample. For example, viruses may be isolated from non-viruses or non-viral components that may otherwise interfere with characterization and/or identification of the virus. Typical examples of such components include cells such as blood cells and/or other tissue cells, and/or any components or fragments thereof. The isolation may, in certain embodiments, further envisage an isolation of different classes of viruses, e.g. allow for an isolation of virus types according to their size, architecture, form etc. The isolation may, in certain embodiments, result in the provision of a collection or layer or accumulation of viruses or sub-classes thereof as defined herein, wherein the viruses are more concentrated than in the original liquid sample. In certain embodiments, said accumulation is present within the context of the original liquid sample, or outside of the context of said original liquid sample. Such a concentrated layer or accumulation of viruses may range from a closely packed dense clump of viruses to a diffuse layer of viruses.

In further embodiments, the present invention also envisages the isolation of cells, e.g. virus-infected cells, from their original liquid sample and/or from other components in said liquid sample, e.g. viruses. This may, for example, include a process of concentration of cells, or specific cell-types such as erythrocytes, within the original liquid sample, whereby significant amounts of the original liquid sample are removed, while cells, e.g. erythrocytes, are not removed. Further envisaged is an at least partial purification of cells from the liquid sample, or from any non-cellular component within the sample. For example, cells may be isolated from non-cellular components that may otherwise interfere with characterization and/or identification of the virus infection of a cell, or of a specific cell type such as an erythrocyte. The isolation may, in certain embodiments, further envisage an isolation of different classes of cells, e.g. allow for an isolation of cell types according to their size, architecture, form etc. The isolation may, in certain embodiments, result in the provision of a collection or layer or accumulation of cells, wherein the cells are more concentrated than in the original liquid sample. In certain embodiments, said accumulation is present within the context of the original liquid sample, or outside of the context of said original liquid sample. Such a concentrated layer or accumulation of cells may range from a closely packed dense clump of cells to a diffuse layer of cells.

In some embodiments, the isolation of the virus from a liquid sample includes the lysis of a cell present in said liquid sample and subsequent centrifugation or filtration, e.g. as defined herein above. Such a cell may comprise viral particles or parts thereof which are thereby released, or the cell may constitute a part of the sample and be destroyed by lysis in order to achieve a separation from virus particles or virions. The term “cell lysis” or cellular disruption refers to a method in which the outer cell membrane is broken down or destroyed in order to release inter-cellular materials such as DNA, RNA, protein or organelles including viral particles or parts thereof or viruses from a cell. Different methods have been developed to lyse cells. The present invention envisages mechanical and non-mechanical lysis methods.

In mechanical lysis, the cell membrane is physically destroyed by using shear force, for example by using a homogenizer, e.g. a high pressure homogenizer, or a bead mill. In a homogenizer, cells in media are force through an orifice valve using high pressure. Disruption of the cell membrane occurs due to high shear force at the orifice when the cell is subjected to compression while entering the orifice and expansion upon discharge. As opposed to the homogenizer, cells are disrupted in the bead mill by agitating tiny bead, e.g. glass, steel or ceramic beads, which are mixed along with the cell suspension at high speeds. Beads may then collide with the cells breaking open their membrane.

In a further embodiment, a virus particle or parts thereof can be also isolated from cells by non-mechanical lysis. A non-mechanical lysis may be a physical, chemical or biological lysis technique. Physical disruption typically uses external forces to rupture the cell membrane. The external forces may include heat, pressure and sound energy. Corresponding eligible methods may hence include thermal lysis, cavitation or osmotic shock. In thermal lysis, cells are subjected to repeated freezing and thawing cycles which causes formation of ice on the membrane leading to its breaking. Cavitation relates to the formation of tiny cavities or bubbles and their subsequent rupture in the cell membrane by reducing local pressure which can be performed by increasing the velocity, ultrasonic vibration, etc. Cells may also be lysed by osmotic shock, wherein the salt concentration surrounding a cell is changed such that the cell membrane becomes permeable to water due to osmosis. Due to the entering of water, the cell swells up and bursts.

In a preferred embodiment, cell lysis is performed chemically. Chemical cell disruption uses lysis buffers to disrupt the cells membrane by changing the pH. Detergents may also be added to the cell lysis buffers to solubilize membrane proteins. In alkaline lysis, an OH⁻ comprising lysis buffer reacts with the cell membrane and breaks the fatty acid-glycerol ester bonds, thereby rendering the membrane permeable. In a further envisaged technique, detergents such as surfactants may be used to disrupt the hydrophobic-hydrophobic interactions between the molecules of the cell membrane. Another envisaged method for lysing cells is enzymatic cell lysis. This approach is based on the use of enzymes such as lysozyme, lysostaphin, zymolase, cellulose, protease or glycanase. In certain embodiments, two or more different techniques may be combined, e.g. mechanical and non-chemical lysis methods may be combined.

In a further embodiment, the viruses, cells or exosomes are isolated or additionally isolated via filtration. For example, the filtration may be performed without previous lysis of cells, e.g. if virus infected or affected cells shall be isolated, or after a cell lysis as described herein has been performed, e.g. if the viral particles per se shall be isolated. Similarly, the filtration may be performed without previous centrifugation of cells, viruses or exosomes, or after a centrifugation as described herein has been performed, e.g. if a centrifugation based pre separation has already been performed. The term “filtration” as used herein generally refers to a separation process based upon the size difference between the suspended particles, e.g. of viruses, cells or extracellular vesicles, in particular exosomes or other cell components/fragments, and the size of the passageways, i.e. pores, present in or on the filter. The filtration is hence designed to size-exclude components within the liquid sample which are larger than viruses or exosomes, respectively, thereby allowing for a separation and, in consequence, isolation of cells or other components from the viruses or exosomes.

A filter may, in typical embodiments, be composed of a filter membrane. According to the present invention a “filter membrane” may be a membrane material comprising single-layer, woven nylon meshes, or being composed of cellulose acetate, polyethylether, nylon, glass fiber or polytetrafluorethylene. Further envisaged are hydrogel, collagen-gel or alike fibrous materials. The membrane may have pores of a range of suitable maximum diameters so as to prevent or allow the passage of viruses, exosomes or in specific embodiments cells of a certain size. The filter membrane may, in preferred embodiments, be filter membrane for microfiltration (pore size of >0.1 μm) or ultrafiltration (pore size of 20 to 100 nm). Alternatively, the filtration function may also be performed by non-classic filters such as silicon nitride layers. Such layers are envisaged to comprise micro-holes, e.g. in the range of 1.5 to 3 μm (diameter) or of about 0.22 μm to 0.45 μm (diameter). It is further envisaged that a filter of biologic origin be used. Suitable examples include filters comprising agarose, hydrogel and/or collagen material.

In some embodiments of the present invention, filtration is performed to size-exclude components within a liquid sample, which are larger than a virus. This may be performed for example by sterile syringe filtration at 0.45 μm or ultrafiltration using a filter with a 10 kDa molecular weight cut-off membrane, e.g. Amicon Ultra-4, to separate virus from the other components released from the cell after lysis, such as proteins or organelles. In further embodiments, filtration is performed to size-exclude components within a liquid sample, which are smaller than a cell.

In this context, exosomes and viruses or virions may be isolated from larger components of the samples, e.g. cells by filtration through a filter membrane having pore sizes of about 0.5 to 1 μm (diameter). By using such a pore size exosomes and viruses will not be retained by the filter membrane, thus pass said membrane and can be collected together with the liquid portion of the sample, whereas larger particles such as cells will be retained at the pores or holes of the membrane.

Alternatively or additionally, a second filtration process may be used to concentrate viruses (virions) and exosomes and/or to separate viruses and/or exosomes from liquid components of the sample. Accordingly, a filtration through a filter membrane having pore sizes of about 15 nm to 25 nm (diameter) may be performed. By using such a pore size viruses and exosomes, which are assumed to have an average diameter of about 30 to 100 nm will be retained by the filter membrane and can thus be collected on the pores of the filter membrane, whereas the liquid portion of the sample passes said membrane. Accordingly, viruses and exosomes may be isolated and/or separated from liquid components of the sample. In certain embodiments, the liquid sample does not comprise viruses and exosomes together. In such a scenario, either viruses or exosomes may be isolated and/or separated from further components of the sample.

In another embodiment, the filtration step may be performed directly on a chip having filtering units downstream of the inlet. It is particularly envisaged that the chip is designed to size-exclude components within the liquid sample which are larger than the virus. In further embodiments, the chip is designed to size-exclude components within the liquid sample which are larger than exosomes. The liquid sample may, in further embodiments, have been processed before usage on the chip, e.g. by lysis procedures as defined herein.

In a particularly preferred embodiment, the present invention envisages a rapid test format that detects the presence of a virus, or the virus load directly from the sample, e.g. a smear or nasal swab, which subsequently dissolved in a suitable buffer as described herein. In a next step, the suspended sample is introduced into the chip, e.g. the channel of a ChannelSlide as described herein, or depicted in the Figures. Cellular components such as cell debris etc. are advantageously retained on the chip by a filter unit, e.g. as described herein. Subsequently, optical tweezers are used to move through the cleaned solution in order to collect a group of viruses. These viruses are subsequently measured, i.e. Raman spectra are obtained as described herein. This approach advantageously allows to collect viruses without the risk of larger particles, e.g. cell debris or cells, falling into the trap and thereby removing the smaller virus particles.

In a further embodiment the cell may be moved or transported within the chip or a subsection thereof or any other part of a microfluidic system as described herein by using focused lasers such as optical tweezers or UV-microbeams.

It is further envisaged that the optical trapping and transportation forces are produced simultaneously by means of an excitation beam of a Raman spectroscopy system and/or a separate laser.

The term “chip” as used herein relates to a silicon unit or silicon-derivative unit, which is capable of separating viruses or exosomes from other components present in a sample as defined above, of isolating viruses or exosomes and of presenting viruses or exosomes to subsequent analysis steps, in particular spectroscopic analyses by means of spontaneous Raman spectroscopy as described herein. In further, alternative, embodiments, the chip is designed to be capable of separating cells, e.g. virus infected/affected cells, from other components present in a sample as defined above, of isolating cells, e.g. virus infected/affected cells, and of presenting cells, e.g. virus infected/affected cells to subsequent analysis steps, in particular spectroscopic analyses by means of spontaneous Raman spectroscopy as described herein. It is preferred that the chip is a Raman compatible fluidic chip.

In certain embodiments, the chip is capable of retaining viruses, exosomes or cells in suitable chambers and allows for analysis of interaction of single cells upon virus infection or exosome treatment and/or treatment of infected cells with antiviral agents as well as for transport and analysis of viruses, exosomes and cells, as well as cultivation of cells. The cultivation and/or analysis functions may, in preferred embodiments, be performed in specific micro-chambers or zones of the chip, which are connected to channel- or passage-structures, e.g. in the form of micro-channels. The transport function may be implemented via the micro-channel(s) and/or main channels, which may split or open into several micro-channels, which in turn end in micro-chambers. Transport of cells, viruses or exosomes into micro-chambers as defined herein may be implemented in various suitable ways. For example, microfluidic techniques as described in more detail below can be used to transport the cells, viruses or exosomes into and out of the chamber. Also the transport of medium, ingredients, antiviral components, lysis reagents etc. may be performed with microfluidic elements such as laminar flows, capillary forces etc. Alternatively, the transport of cells, viruses or exosomes, as well as their arrest at specific locations, e.g. in a micro-chamber, may be performed with electromagnetic forces, preferably with focused lasers such as optical tweezers or UV-microbeams as defined herein below. In further embodiments, electromagnetic gradients between electric poles, i.e. plus and minus, may be sued. Further envisaged are induced electrical fields, or centrifugal forces which are applied to the cells, viruses or exosomes. In a preferred embodiment, a correspondingly designed fluidic channel may be have the form of a spiral with chambers located at the outside, designed to receive the cells, viruses or exosomes upon application of the mentioned forces, e.g. centrifugal forces.

A chip may comprise an inlet, e.g. for injection of liquid samples, which may be injected into the inlet via a syringe or the like, as well as a multitude of micro-chambers and corresponding micro-channels, e.g. between 2 to 1000 separate micro-chambers and corresponding micro-channels, which may be arranged in any suitable manner to allow for a transport, analysis and optionally cultivation of cells, viruses or exosomes. For example, the micro-chambers may be located in a star-like manner around a central channel structure. Alternatively, the micro-chambers may be arranged at both sides of street-like oriented main-channel. Also envisaged is a ring-like or spiral-like main-channel with micro-chambers at both sides. These channels preferably are used in embodiments in which centrifugal forces are applied. It is preferred that the micro-chambers are used for the enrichment of of cells, viruses or exosomes, e.g. via transport processes or arresting procedures as described herein. In specific embodiments, some of the micro-chambers are designed for cultivation of cells, e.g. by comprising cultivation medium, or by having a connection to a channel transporting cultivation medium to the cells.

In a specific embodiment, a composition comprising virus particles, exosomes or cells is applied to a filtering unit as described. Accordingly, particles larger than a virus or exosome may be retained be the filter, whereas a virus or exosome can pass unhindered and enter micro-chambers of the chip downstream of the filtering unit. The virus or exosome, or groups thereof, may subsequently be enriched in said micro-chambers.

In particularly preferred embodiments the exosomes are enriched in a channel of the chip as defined herein.

In case the liquid sample comprises either virus material or exosome material, a differentiation between both components is not required. Should both materials be present in a liquid sample, an immunocapturing step as defined herein may be performed.

In further specific embodiments, the chip comprises an antiviral agent exposure unit capable of and designed for determining the susceptibility of a cell, e.g. a virus infected/affected cells to an antiviral agent. The antiviral agent exposure unit is designed as a micro-chamber which may be located in a specific part of the chip. In an embodiment, the antiviral agent exposure unit of the chip may comprise one or more micro-chambers comprising an antiviral agent or a combination of antiviral agents. Accordingly, the micro-chamber comprises a suitable amount of an antiviral agent or a combination of antiviral agents, e.g. one of the antiviral agents as mentioned herein, or is connected to a reservoir or channel transporting the antiviral agent to the micro-chamber. In a preferred embodiment, said antiviral agent or said combination of antiviral agent is lyophilized. The term “lyophilized” refers to the state of an antiviral agent, wherein it underwent a freeze-drying process to remove water from the antiviral agent after it is frozen and placed under a vacuum. It is envisaged herein that said lyophilized antiviral agent is activated upon contact with a liquid, which is, for example, provided to the chambers via the inlet of the chip. When the isolated cells come into contact with the activated antiviral agent, the susceptibility to said antiviral agent may be determined by recording and comparing Raman spectra prior and subsequent to the exposure to the antiviral agent as described herein. In further embodiments, the determination of a susceptibility to the antiviral agent may be performed with the support of Raman spectra databases as described herein. A comparison with data sets in the database may be performed, for example, during or after the determination. The cells to be analyzed may be derived directly from samples, or may have been prepared, processed or cultured before determination, or have been enriched before determination as described herein.

In a specific embodiment the chip comprises μm sized channels. Alternatively, the chip comprises μm sized channels with an integrated filtering unit. In a further alternative embodiment the chip comprises μm sized channels with an integrated filtering unit and an antiviral agent exposure unit capable of determining the susceptibility of cells to an antiviral agent.

The chambers and channels of the chip may have any suitable size, preferably in the μm range. It is accordingly envisaged in a preferred embodiment to provide micro-chambers with a height of about 10 to 300 μm and diameter of about 10 to 500 μm. The filter area could cover an area of about 1 mm² up to 1 cm². The main channel may preferably have a width of about 50 to 500 μm, a height of about 50 to 200 μm and a length of about 100 μm to 1 cm or more, depending on the number of micro-chambers. Side channels connecting the main channel and the micro-chamber may preferably have a height of about 50 to 500 μm and a length of about 50 to 80 μm with a width of about 10 to 30 μm.

The chip may be composed of any suitable material. It is preferred that the material is at least partially translucent and allows for spectroscopic analyses by means of spontaneous Raman spectroscopy. The bottom of the chamber is preferred to be composed of Raman compatible material. Suitable examples include quartz glass, CaFl₂ (calcium fluoride) glass or borosilicate glass. It is particularly preferred that the material is translucent. In further preferred embodiment, the chip or parts or it are translucent. Also envisaged are semi-translucent materials. In preferred embodiments, the chip is fabricated from glass by conventional direct laser structuring, powder or sandblasting, or photostructuring. Further eligible materials for the construction of the chip are thermoplastic polymers, such as polymethylmetacrylate (PMMA), polycarbonate (PC), polystyrene (PS), Topas, Zeonor, or Zeonex. Also envisaged is PMDS, which is optically transparent and biocompatible. The processing technique varies with the material used for the fabrication of the chip. For example, thermoplastic polymers can be process via injection molding, thermoforming, hot embossing, laser machining, or precision mechanical machining. The processing techniques are known in the art and can accordingly be applied by a skilled person. In addition, the chip may be coated, for example, with one or more virozides or antiviral substances.

The chip unit may be equipped with a filter membrane as defined herein above. The filtration membrane is typically located downstream from the inlet. The chip may, in one embodiment, comprise a filter membrane which is capable of retaining cells or larger non-viral particles or cell fragments and thus prevents their entering into inner parts of the chip, in particular into the micro-chambers, while letting pass viruses and/or exosomes. In further embodiments, a similar filtration membrane may be used to enrich cells and to subsequently allow the cells to enter the inner parts of the chip. Accordingly, smaller particles such as viruses or exosomes may be excluded. The filter membrane may be positioned at any suitable central location within the chip to allow for an efficient filtration of samples. Preferably, a filter membrane may be provided in the initial or opening segment of a main channel as defined herein, thus allowing the passage of cells, viruses or exosomes via the main channel to micro-chambers as defined herein. Alternatively, the filter membrane may be provided above or in the vicinity of the micro-chambers and thus allow for a direct loading of said chambers through the pores of the membrane. In one embodiment, a filter membrane is provided which comprises a suitable hole or pore above a micro-chamber and hence allows for loading of each of said chambers with cells, viruses or exosomes separately. It is particularly preferred that the filter membrane is provided as integral part of the chip as defined herein.

In further specific embodiments, the filter membrane allows for a removal of non-elected entities from the micro-chamber zones of the chip after the filtration process is finished. For example, the filter membrane may be designed as separate layer on top of a chip comprising a multitude of micro-chambers. After the sample has been filtered through said filter membrane and the elected entities, e.g. cells, viruses or exocomes have entered the micro-chambers, said layer is removed, e.g. by a sliding mechanism. Alternatively, the chip comprising the elected entities in the micro-chambers may be moveable and thus be separated from the filter membrane after the sample was filtrated and the elected entities have entered the micro-chambers.

The chip may, in further embodiments, also comprise a control checkpoint, which typically resides downstream of the filtration unit to check the status and function of the filtration process or filter membranes. In specific embodiments, a micro-channel or micro-chamber located below the filtration unit may be filled with the filtered sample. The termination of this process may, for example, controlled via the presence of semipermeable membranes which are closed once they are in contact with liquids. Also envisaged is an optical and electronic detection mechanism, e.g. via CCD cameras etc., which detects/monitors the filling status of the micro-channel or micro-chamber located below the filtration unit.

Components that are able to pass through the filtration unit and the control checkpoint, may enter the chip, e.g. via one or more of the channels as described herein. Particles or components which are size-excluded by the filter membrane do not enter said channels and are retained on the filter membrane. Further envisaged is a waste or outlet located downstream of the channel, which may be used to evacuate the filtered liquid from the channel for downstream measurements.

In preferred embodiments, the chip is connected to, or integrated into, or part of a microfluidic system. The term “microfluidic system” as used herein relates to a device allowing the precise control and manipulation of fluids that are constrained to small, preferably sub-millimeter scales. Typically, a microfluidic system implements small volumes, e.g. in the range of nl, or pl, and/or it may implement an small overall size. Furthermore, a microfluidic system according to the present invention may consume a low amount of energy. In a microfluidic system according to the present invention effects such as laminar flow, capillary flow, specific surface tensions, electrowetting, fast thermal relaxation, the presence of electrical surface charges and diffusion effects may be implemented and/or used. In certain embodiments, a microfluidic system may have connections with external sources or external elements, e.g. the separation or reservoirs or vessels for reuse purposes may be possible. It is preferred that the system is, at least partially, based on capillary forces. In addition or alternatively, active elements such as micropumps or microvalves may be used. A microfluidic system as envisaged by the present invention may comprise several modules which may be connected by channels. It may further comprise a reservoir for cells and a reservoir for fluids or buffers etc. For the performance of the analysis of a cell, virus or exosome the microfluidic system may comprise a chip with a network of channels, as described herein, which is connected to a Raman spectroscopy system.

The microfluidic system may, in specific embodiments, also comprise zones or modules where nucleic acids can be isolated and analysed, or a module which is configured to allow antibody binding, or an array of microwells allowing for contacting of cells, viruses or exosomes with a substance, or which allows for cultivation of cells or any other suitable module or element. Preferably, said channel or zone is configured to slow down liquid movements to allow for optical/spectral analysis of the cells, exosomes or viruses. In further embodiments, hydrogels, collagen gels or other material which slow down cells, viruses or exosomes may be used in the system, e.g. within a meshwork of fibers.

Furthermore, the microfluidic system may comprise an electronic or computer interface allowing the control and manipulation of activities in the system, and/or the detection or determination of reaction outcomes. In another specific embodiment of the present invention said microfluidic system may be an integrated microfluidic system. The term “integrated microfluidic system” as used herein refers to the compactation and resizing of the chip in the system, as well as the system itself, e.g. comprising all necessary connections, zones and, optionally, also necessary ingredients within container-like form. The integrated microfluidic system may, for example, have the form of a cartridge and, thus, be entirely closed, or partially closed allowing the introduction of samples, ingredients etc. via resealable inlets. As a cartridge, the system may further be replaceable in an uncomplicated manner. Accordingly, the cartridge may be connected to surrounding units by interfaces which are capable of single step disconnections or simple disruptions. The integrated microfluidic system may further be equipped with alignment structures for optical detection or illumination/stimulation devices. Such a cartridge systems allows for a safe handling of samples which prevents infection or contamination of lab technicians or laboratories. Furthermore, the cartridge approach facilitates an easy and comfortable cleaning, sterilization of the apparatus and/or preparation for further samples to be analysed.

Further envisaged is a unit allowing for the recognition of sample- or ingredient-associated information, e.g. recognition by a scanner of a bar code or matrix codes indicating the sample origin, patient identity, sampling time, sampling location, type of sample etc., or the identity of provided ingredients, the manufacture date etc. In specific embodiments, the recognition may be implemented via a unit for contactless communication with a base station outside of the system or as part of the control module of the system, which comprises a corresponding reader. Examples of suitable contactless communications units are an RFID (radio frequency identification) unit, preferably a NFC (near field communication) unit, a Bluetooth unit or an ID-chip unit. In a typical example, the sample may be tagged with an RFID chip and accordingly be recognized by a suitable RFID reader. Also envisaged is the presence of an interface to a detection unit allowing the electronic or optical determination of analysis outcomes, object/cell positions etc. The chip may further be designed for storage and documentation purposes, e.g. have a geometrical or design element which facilitates storage in a box, refrigerator or safe.

In some cases, the virus to be analyzed can be cultured by infecting cells, for example, virus may be added to a screw-cap tube containing a cell monolayer and a suitable medium. Subsequently, virus may be released into the supernatant of said cell culture. As such, in some embodiments the supernatant of a cell culture of infected cells can be applied to a chip directly without involving further isolation steps.

In another aspect the present invention relates to a method for monitoring a viral infection in a cell or group of cells. In a preferred embodiment, the viral infection is monitored in a cell or group of cells in a cell culture, e.g. a cell culture as defined herein. The term “monitoring” refers to the observation of a disease, condition or one or several medical parameters over time. In the present invention, monitoring relates to the observation of viral infection of cells, viral propagation in a cell or group of cells or a cell culture over time, the dynamics of viral infections, e.g. spectral analysis of cells over time, the effect of additional compounds administered to the cells, the effect of changes to condition under which the cells are cultivated, the effects of superinfections or second infections, e.g. with the virus or a different virus, the observation of morphologic changes to cells during the infection, e.g. apoptosis events etc. In one embodiment, the viral load may be used as marker of dynamic changes over time. For example, the viral load levels may be quantified to facilitate prediction about disease progression, or to predict a response to an antiviral agent and monitor the effects of such an administration. This approach may also allow the analysis of different stages in a viral infection, virus entry processes, pathologies induced by viruses and/or the cellular viral reservoir. Quantification may, for example, be based on spectroscopic analysis as described herein, e.g. Raman spectroscopy of cells, extracellular liquids or supernatants. Alternatively, or in addition, it may be based on the detection of viral DNA/RNA copies per ml of sample, e.g. blood plasma, supernatant of cell culture medium etc. In one example, the total amount of viral RNA from a cell or a group of cells, or in a cells or group of cells in a cell culture may be obtained and thus, thereby monitoring viral infection.

In one particular, embodiment, the cell or group of cells to be monitored may be suspected to be infected by a virus, is derived from a virus infected/affected cells or is a cell, which has been deliberately infected with a virus. In a further embodiment, the cell or group of cells is derived from a cell culture, e.g. as described herein above, or from a patient's sample, e.g. as defined herein above. Such cells, in order to be monitored, need to observed for a certain period of time, e.g. several minutes, 30 minutes, 1, 2, 3, 4, 5, 6, 7, 8, 9, 10, 11, 12, 15, 18, 24 h, 2, 3, 4, 5, 6, 7, 8, 9, 10, 11, 12, 13, 14 days, 3, 4 weeks, 2, 3, 4, 5, 6, 7, 8 or more months or any period in between the mentioned values. For cells to monitored for a longer period, e.g. more than 30 min to 48 h or longer, the cells may preferably be cultured or provided with nutrients, e.g. be kept in a cell culture. The nutrients, growth factors, pH etc. necessary for the specific cell type may be adapted to said cell type, e.g. in accordance with suitable literature references or suitable knowledge of the skilled person.

The term “deliberate infection with a virus” refers to an experimental approach in which a predefined type of cells, preferably in a predefined number, e.g. 50, 100, 200, 300, 400, 500, 1000 cells or more are contacted with a virus. The virus may, for example, be provided in different multiplicity of infection (MOI) ratios of virus appropriate to infect the cells. As the MOI increases, the percentages of cells infected with at least one viral particle also increases. For example, the MOI may be in the range of 0.1 up to 8.0 (— 100% infected cells). The virus may be derived from patient's sample, or from any other suitable source. It may be a provided in predefined number, concentration, processed state, in a suitable buffer or pH, or under any other suitable condition necessary for an effective infection. The cells may be contacted with the virus in a group or batch procedure, or they may be contacted individually or in small groups of cells. The deliberate infection may be performed once, or more than one time, e.g. 2, 3, 4, 5 times, e.g. over a certain period of time, e.g. with a second infection after 6 h, 12 h, 24 h, 2 days etc. In as specific embodiment, the deliberate infection with a virus is performed at any time point or stage during and/or before the monitoring and/or may be repeated at least once.

It is particularly preferred that during an approach for deliberate infection of cells, the cells are monitored during the entire phase of infection, starting with non-infected cells and including all stages of infections, or sub-portions thereof.

In another embodiment, the sample derived cell or group of cells or the cell culture derived cell or group of cells is or has been treated prior or during the monitoring of the viral infection with an antiviral agent. The antiviral agent may be an antiviral agent as defined herein above. In further embodiments, the cell or group of cells may be treated with or administered with a vaccine, antibody, antiviral development candidate or any other substance which is considered to affect a virus infected/affected cell or could affect the virus infection of a cell.

In yet another embodiment, the method of monitoring a viral infection comprises recording at least one Raman spectrum by means of Raman spectroscopy of a virus in the cell or group of cells, or of a virus infected/affected cell or group of cells, preferably as described herein. The Raman spectrum may further be statistically analysed and processed as defined herein.

It is envisaged herein to record at least one Raman spectrum during the monitoring by means of Raman spectroscopy of a virus, or a virus infected/affected cell or non-infected cell. In certain embodiments, a plurality of Raman spectra of a virus, e.g. group of viruses, or a virus infected/affected cell or non-infected cell may be obtained in order to draw more accurate conclusions on the identity and properties of a virus, or the virus infected/affected cell by means of statistical analysis. In preferred embodiments, at least 2, 3, 4, 5, 6, 7, 8, 9, 10 or more Raman spectra of a virus, e.g. group of viruses, or virus infected/affected cell are recorded. The plurality of spectra may either be obtained for a virus, e.g. a group of viruses or for a single cell, e.g. one spectrum may be obtained for one cell, or a group of cells. It is particularly preferred to obtain spectra for viruses or single cells, e.g. via the use of optical traps as mentioned herein.

In specific embodiments the Raman recording is performed prior and/or subsequent to the viral infection. “Prior to viral infection” refers to the acquirement of a Raman spectrum before a virus comes into contact with a cell, a group of cells or cell culture, or before viral infection of cells or viral dissemination has occurred. There is no time restraint or limit as to the acquirement of such Raman spectra. The information may, in certain embodiments, have been obtained at any point of time in the past and also be derived from databases or previously recorded spectra or be additionally compared or supplemented with information from previously recorded spectra or database information. “Subsequent to the viral infection” means obtaining a Raman spectrum after a virus has come into contact with a cell, a group of cells or cell culture for a specific period of time, or viral infection of cells or viral dissemination has occurred in the cells, group of cells or cell culture. Accordingly, in certain embodiments the cells may be spectroscopically analyzed before the infection is performed and/or at least at one time point after the infection, e.g. after 5 min, 10 min, 30 min, 60 min, 2, 3, 4, 5, 6 h or more.

The monitoring of a virus infection over time advantageously allows to draw conclusion on the progression and spread of the viral infection. As such, it is preferred to record Raman spectra several times subsequent to the viral infection. In one embodiment, the recording is performed 2, 3, 4, 5, 6, 7, 8, 9, 10 times or more often subsequent to the viral infection. In preferred embodiments, the recording is performed in fixed time intervals according to a predetermined schedule. For example, the recording may be performed at definite lengths of time such as every 2, 4, 8, 16, 20, 24 h, days or weeks subsequent to a viral infection. A “predetermined schedule” as used herein refers to a plan that gives a list of conditions under which the recording is to be performed. It is preferred that identical conditions are used for the recording, since this may reduce bias in the measurements. The conditions may, for example, refer to sample source, sample amount, sample preparation, settings of Raman spectroscope, recording time and quantity, and the like.

In a further main aspect the present invention concerns an in vitro method for analysing exosomes in a liquid sample of a subject comprising: (a) isolating exosomes from the liquid sample; (b) analysing said exosomes spectroscopically by means of spontaneous Raman spectroscopy; and (c) obtaining a Raman spectrum for said exosomes.

The term “exosome” relates to an extracellular vesicle as defined above and means a vesicle originating as ESCRT-dependent invaginations of early endosomes that are released into circulation upon fusing of the resultant multi-vesicular bodies (MVBs) with the plasma membrane. In certain embodiments, the term also refers to similar vesicles, which are known as ectosomes, exomeres or oncosomes (see, for example, Rojalin et al., 2019, Front. Chem., 7, 279 for further details). In further embodiments, an exosome as defined in the present invention may have the format and properties of a microvesicle. The size of an exosome according to the present invention typically ranges from about 40 to about 150 nm. In certain embodiments, exosomes may have larger sizes up to 500 nm, 750 nm or 1000 nm. In case such large exosomes are concerned, filtration, isolation, separation or quantification procedures may be adjusted, e.g. with respect pore sizes, filtering steps etc. Exosomes are typically generated in the endosomal compartment of cells by invagination of the cell membrane (endocytosis) and the subsequent formation of intracellular vesicles, which are released toward the extracellular space in response to different signals (exocytosis). The signals for exosome release may be stressful cellular conditions (e.g. hypoxic situations), pH or ionic alterations, viral infection, ischemia, phosphatidylinositol 3-kinase and loose cell-to-cell adhesion. As a result of the cell membrane budding, the molecular composition varies depending on the origin cell. As such, exosomes are typically surrounded by a lipid bilayer with characteristic and cell-specific membrane proteins, such as tetraspanins, integrins, various intercellular adhesion molecules or the major histocompatibility complex. Moreover, they contain a variety of molecules, such as lipids, proteins and nucleic acids, including messenger-RNA (mRNA) and microRNA (miRNA). The identity and amount of these molecules may vary depending on the origin and formation process of the exosome. The specific biochemical and molecular composition of exosomes may thus serve as diagnostic marker and may accordingly be used for diagnostic or differential detection approaches, e.g. in addition to a spectroscopic analysis as described herein above.

The present invention thus envisages the analysis of exosomes with respect to the presence of components such as mRNA, miRNA, DNA, proteins, e.g. membrane-spanning proteins, enzymes, or heat shock proteins. According to the features of the Raman analysis as described herein, pattern of exosomes are detected. The pattern are based on the analysis of the sum of all molecules present. The methodology thus advantageously allows to distinguish between the mentioned entities, e.g. exosomes of different origin and/or associated to specific health states or diseases, on the basis of said pattern.

The in vitro method for analysing exosomes in a liquid sample according to the present invention comprises as step a) an isolation step of an exosome from the liquid sample.

As used herein, the term “isolation” or “isolating” in the context of exosomes refers to a process of removing or otherwise setting apart or separating exosomes from their original liquid sample and/or from other components in said liquid sample. The term may further relate to a process of concentration of exosomes within the original liquid sample, whereby significant amounts of the original liquid sample are removed, while exosomes are not removed. The isolation of exosomes may be performed by any suitable means known to the skilled person and at any suitable point of time during the analysis. The isolation may, for example, be performed as pre-analytic step before the exosome is spectroscopically analyzed as described herein. Accordingly, the isolation may be performed at a different site, with a non-connected module or device or, in the alternative, may be performed within the device or an integral part of the system as described herein. The term “isolation” may, in certain embodiments, further include an at least partial purification of exosomes from the liquid sample, or from any non-exosome or non-exosome component within the sample. For example, exosomes may be isolated from non-exosome or non-exosome components that may otherwise interfere with characterization and/or identification of the exosome. Typical examples of such components include cells such as blood cells and/or other tissue cells, and/or any components or fragments thereof. The isolation may, in certain embodiments, further envisage an isolation of different classes of exosomes, e.g. allow for an isolation of exosomes types according to their size, surface, presence of markers such as sugar or protein markers etc. The isolation may, in certain embodiments, result in the provision of a collection or accumulation of exosomes or sub-classes thereof as defined herein, wherein the exosomes are more concentrated than in the original liquid sample. In certain embodiments, said accumulation is present within the context of the original liquid sample, or outside of the context of said original liquid sample. Such an accumulation of exosomes may range from a closely packed dense clump of exosomes to a diffuse layer of exosomes.

In a preferred embodiment, the isolation is performed on a chip designed to separate cells or cellular components from the liquid phase of the sample, as defined herein above. Accordingly, in a set of preferred embodiments, the isolation may be performed directly on a chip having filtering units downstream of the inlet. It is particularly envisaged that the chip is designed to size-exclude components within the liquid sample which are larger than exosomes. The liquid sample may, in further embodiments, have been processed before usage on the chip, e.g. by lysis procedures. In certain embodiments, the isolation is performed via filtration steps, e.g. as described above in the context of a chip. Alternatively or additionally the isolation of exosomes is performed via immunocapture of the exosomes. The term “immunocapture” as used herein relates to an immunobinding and subsequent capturing of exosomes via surface markers present on the exosomes. In preferred embodiments, said surface markers are proteins which are present on the surface of exosomes, but are missing on cells surfaces or the surface of viruses or virions. Examples of suitable markers include Rab5b and CD63, which are present on exosomes, endosomes or lysomes. Since endosomes or lysosomes are intracellular entities and since the proteins are typically not shed or recycled, they are suitable as differential exosome markers. Further suitable and envisaged markers include carbonic anhydrase (CAIX), CD9, Alix, TSG101, syntenin and CD81. The biomarkers may be used alone or in any combination. For example, Rab5b, CD63, CAIX, CD9, Alix, TSG101, syntenin and CD81 may be used alone or in a combination of any one, two, three, four, five, six, seven or eight of this group. Preferred are combinations of CD9 with other markers as mentioned above, of CD81 with other markers as mentioned above and of CD63 with other markers as mentioned above. Further preferred are combinations of CD9 with CD81, CD9 with CD63, CD63 with CD81, or CD9 with CD81 and CD63.

The immunocapture may be performed with and/or combined any suitable platform technology. The binding to surface makers as mentioned above may, for example, be performed in the context of the use of microbeads or in the form of an extraction by using immunomagnetic beads. This typically leads to the magnetic labelling of the exosomes. Subsequently, the labelled exosomes may be separated, e.g. with a magnetic field column technology allowing for a retaining in the column whereas other components are run through. After the magnetic field is removed from the column, the exocomes can be eluted and obtained. Also envisaged is the use of ELISA technique. The capturing may be carried out with suitable binders such as antibodies, e.g. polyclonal or monoclonal antibodies. The isolation may preferably be combined with additional purification steps such as size exclusion filtration and/or centrifugation etc. Unspecific reactions may further be prevented by cleaning the sample with quantitative binding of nonexosome material. Further details would be known to the skilled person or can be derived from suitable literature sources such as Logozzi et al., 2020, Methods in Enzymology, Volume 645, page 155-180.

In further embodiments, the exosomes are isolated via centrifugation procedures. These centrifugation procedures may comprise differential centrifugation, ultracentrifugation or density gradient centrifugation.

Differential centrifugation allows to isolate exosomes based on their density and size differences from the liquid sample. Typically, ultracentrifugation may be used in combination with sucrose density gradients or sucrose cushions to float the low-density exosomes away from other vesicles and particles. For ultracentrifugation forces up to 200,000 g may be used.

The centrifugation steps may be performed previous to further analysis steps. They may, in certain embodiments, be performed in specific centrifuges which may be located at a different site in comparison to the device which is used to perform the spectroscopic analysis. Alternatively, centrifugation procedures may be combined with further method steps, e.g. in a combined system or modular framework. Isolated exosomes, e.g. via centrifugation or other means, may subsequently be entered into a chip structure as described herein.

In further embodiments, the exosomes may be isolated by chromatography, ultrafiltration separation, or PEG-based precipitation.

Chromatography approaches may, in particular, comprise size exclusion chromatography (SEC). Size exclusion chromatography typically uses a stationary phase consisting of porous resin particles. Molecules smaller than the isolation range (e.g. >35 nm or >70 nm) are slowed because they enter into the pores of the stationary phase. Larger particles which cannot enter the pores flow around the resin and may be eluted from the column earlier. Molecules and small particles that enter the pores have longer retention times and elute later.

Ultrafiltration can isolate exosomes based on their defined molecular weight or size using membrane filters. The ultrafiltration may be performed with several sub-steps including a normal prefiltration, followed by a tangential ultrafiltration for proteins smaller than 500 kD and a final ultrafiltration with a 0.1 μm pore size.

PEG-based precipitation makes use of the fact that PEG is a water-excluding polymers which can tie up water molecules and forces less soluble components out of solution. Liquid samples may accordingly be incubated with a precipitation solution containing PEG, leading to a precipitate containing exosomes, which can subsequently be isolated by means of low-speed centrifugation or filtration.

In further embodiments the isolation may be performed with microfluidic systems or tools. Examples of such isolation approaches which are envisaged by the present invention are acoustophoresis, electrophoresis-driven filtration, dielectrophoresis, magnetophoresis, on-chip centrifugation, inertial lift force, viscoelastic flow, microfluidic filtration and microfluidic immunoaffinity. These exosome isolation and purification methods may be performed on a chip as described herein and/or in a microfluidic device or unit as described herein. For example, on-chip centrifugation may be carried out with centrifugal micro-hydrodynamics on a chip, preferably a chip comprising microchannels, a serpentine inlet channel, a microfluidic separation channel and two outlets. The chip may subsequently be rotated to exert centrifugal, Coriolis, buoyance, and hydrodynamic drag forces thus allowing for a separation of exosomes.

The acoustophoresis isolation is based on the generation of acoustic waves such as BAW and SAW waves. It is particularly preferred to use Standing Surface Acoustic Waves (SSAW). Further details may be derived, for example, from Wu et al., 2017, Proc. Natl. Acad. Sci. 114, 10584-10589.

Microfluidic filtration approaches are typically cased on the use of nano-filters, nano-porous membranes, or nanoarrays which are usually used in microchannels to separate particles based on their size. The method makes use of nanofiltration and centrifugation steps, e.g. a chip or microfluidic device is spun and the liquid sample may pass through different nano-filters allowing for a concentration of exosomes. Further envisaged is the trapping of exosomes on ciliated micropillars (see also Li et al., 2019, APL Bioeng. 3, 011503).

A further approach is based on inertial lift forces, which can be used to displace exosomes laterally across microchannels which a sufficient flow rate and velocity differences between exosomes and fluid. Exosomes can accordingly be moved across the channel. The use of additional beads may further be considered to increase the effect.

Yet another approach envisaged by the present invention is use of viscoelastic flow, where elastic lift forces are exerted by a viscoelastic medium to the exosomes. To create the viscoelastic medium, different polymers, such as diluted (low concentration 0.1% w/w) poly-oxyethylene (PEO), can be used. The PEO polymer typically makes the fluid highly viscoelastic and causes an imbalance in the first normal stress difference across the microchannel. This imbalance creates an elastic force proportional to the volume of exosomes. As a result, bioparticles can be positioned laterally across the width of the microchannel based on their volume.

A further approach is immuno-affinity capturing which is implemented in a microfluidics system by modifying a microchannel surface with antibodies and the use of affinity particles or magnetic beads. The use of an external magnetic field may separate exosomes from other components.

The spectroscopic analysis of the exosome in step b) by means of spontaneous Raman spectroscopy is performed as described above. Further, obtaining a Raman spectrum for the exosomes in step c) is performed as described herein above.

In a specific embodiment the method additionally comprises as step d) a step of quantification of the isolated exosomes. As used herein, the term “quantification” relates to the determination of the number of exosomes in a liquid sample, e.g. the liquid sample to be analysed according to the present invention. The quantification may typically take place in a confined volume and/or in a defined area, e.g. of the chip as described herein. For example, the quantitation of exosomes may be performed in one or more the micro-chambers, or channels etc. of the chip as described herein, i.e. after the sample has been filtered and the exosomes have been isolated. The quantification may be carried out according to any suitable means. The quantification may be performed on the basis of characteristic physical properties of exomes, in particular size, mass and density. Alternatively, the quantification may be based on the use of membrane proteins present on the surface of the exosomes.

In preferred embodiments the quantification is performed with the help of an optical trap as described herein. For example, a spectroscopic measurement of exosomes may be performed over a defined period of time, allowing fora determination of the number of exosomes detected.

Further examples of suitable quantification procedures, which may also be used as calibration methods for a spectroscopic quantification approach as described above, include the ELISA-based Immunoaffinity capture (IAC) assay, the nanoparticle tracking analysis (NTA), the asymmetrical-flow field flow fractionation (AF4) coupled with multidetection assay, the dynamic light scattering (DLS) assay and the surface plasmon resonance (SPR) assay or nanoplasmon-enhanced scattering assay. The methods are preferably performed in a different module or with a different device, or may be connected to the currently envisaged chip via a microfluidic integration, e.g. as connected entity.

The quantification approach may further be based on exosome surface markers as mentioned above in the context of the immunecapture isolation procedure. These markers may be used for a quantitative binding to exosomes, thus allowing for a quantification of the particles. Further information would be known to the skilled person or can be derived from suitable literature sources such as Grimolizzi et al., Sci Rep., 2017, 7(1): 15277; Sitar et al., Anal Chem., 2015, 87(18): 9225-9233; or Huang et al., BMC Genomics, 2013, 14(1): 319.

In a particularly preferred embodiment the in vitro method for analyzing exosomes according to the present invention comprises the determination, on the basis of the obtained Raman spectrum as defined herein above, whether a subject is affected by a disease. The subject may be a mammal, e.g. a cow, sheep, dog, cat, monkey, rat, mouse, horse or, preferably a human being. The performance of the analysis accordingly allows to distinguish between a healthy or normal state of a subject and a diseased state, i.e. a state in which the subject is affected by a disease, by analyzing exosomes derived from liquid sample of the subject. The method according to the present invention allows, in a specific embodiment, to determine whether a subject is affected by a disease by analyzing the subject's exosomes and comparing the obtained Raman spectrum with a Raman spectrum of exosomes obtained from an independently defined healthy subject. Deviations in the registered spectra allow the conclusion that subject is affected by a disease.

In an alternative embodiment, the comparison of the Raman spectrum obtained from subject may be performed with a reference Raman spectrum. This may be a spectrum obtained from one or more independently examined healthy subjects, or a spectrum from independently diagnosed diseased subjects or both. The reference spectrum may preferably be present in a database and be derived from said database. The present invention also envisages the use of more than one reference spectrum, e.g. 2, 3, 4, 5, 10, 15 or more spectra. Further envisaged is the comparison with Raman spectra obtained from a specific subject at different time points, e.g. an initial spectrum, followed by a subsequent spectrum obtained after 1 week, 2 weeks, 1 months, 2 months, 3 months, etc, 1 year, 2 years etc. These subsequent spectra may further be derived from a database as described herein. The spectra and subsequent spectra may, for example, be derived during a therapeutic treatment of a subject, e.g. after certain periods of time, thus allowing for a monitoring of the treatment success.

The term “disease” as mentioned herein relates to any type of disease which is detectable via composition changes of exosomes. Examples of such diseases include cancer, neurodegenerative diseases, diabetes mellitus and infections, in particular virus infections. In specific embodiment the disease is cancer such as non-small cell lung cancer, gastric cancer, oral squamous carcinoma, neuroblastoma, bladder cancer, melanoma, breast cancer, pancreatic cancer, glioblastoma. It is particularly preferred that the cancer is an early-stage cancer, e.g. a cancer which cannot or not easily be detected with standard cancer diagnostic approaches. Also preferred is the detection of metastatic cancer forms.

In further specific embodiments the disease is a neurodegenerative disease.

Since exosomes are assumed to cross the blood-brain-barrier, they can be used as diagnostic tool neurodegenerative diseases including Alzheimer's disease or multiple sclerosis (MS). Without wishing to be bound by theory, it is assumed that in Alzheimer's disease exosomes comprising amyloid beta-proteins and/or tau proteins are generated, which can be derived from a subject's liquid sample and be detected with a Raman based spectroscopic analysis according to the present invention.

In a further specific embodiment the disease is diabetes mellitus. The diabetes mellitus may be a type 1 or type 2 diabetes. Exosomes of subject affected by diabetes show a high degree of specific miRNAs and insulin auto-antibodies.

The presence of a viral infection via the existence of virally induced exosomes may also be detected with a method of the present invention as defined above. Corresponding details are provided herein in the context of the detection of viruses and virus infected cells.

In a further specific aspect the present invention relates to a method of monitoring the antiviral treatment effect in a virus infected/affected cells or group of cells, e.g. as defined above. Further envisaged is a method of monitoring an antiviral treatment effect or by analysing exosomes, preferably exosomes derived from liquid biopsies of a patient. An antiviral agent, in particular a natural substance as defined herein, may be used. In a preferred embodiment, the antiviral treatment effect is monitored in a virus infected/affected cell or group of cells in a cell culture. The monitoring is preferably performed via spectroscopy analyses as defined above. In addition, periodic sample or serological studies during an antiviral treatment may be performed to assess for adequate primary response to the antiviral treatment, treatment-related side effects, achievement and maintenance of treatment endpoint, and the emergence of antiviral resistance. In certain embodiments, a recording of Raman spectra is performed 2, 3, 4, 5, 6, 7, 8, 9, 10 times or more often subsequent to the antiviral treatment, preferably in fixed time intervals such as every 5, 10, 20, 30, 60, 75, 90, 120 min, 3, 4, 5, 6 h etc. It is further particularly preferred that the recording is performed previous to the antiviral treatment and/or subsequent to it. This allows for sets of comparable data which may be used to understand the dynamics of infections in the context of antiviral activity, or the quality and speed of an antiviral response of an infected cell. In further specific embodiments, control groups with cells which are not virus infected/affected are treated with the antiviral agent as described herein. These control groups allow for an independent measurement of the physiological changes to the cells upon exposure to the antiviral agent.

In the context of the present invention, it is preferred that identical conditions are used when recording Raman spectra of samples to reduce background noise and a potential bias in the measurements. In certain embodiments, these conditions may also be controlled in the context of culturing cells, e.g. in a cell culture as defined herein. Accordingly, any suitable parameter describing the function, stage, or prospect of a cell may be controlled. For example, in one embodiment, the growth and/or the natural status of said cell or group of cells is controlled. In a preferred embodiment, this control comprises, within a cell culture or any receptacle where non-infected cells, cells suspected to be virus infected/affected, cells which are virus infected/affected or have been deliberately virus infected or have been deliberately virus affected or cells, which have been treated with an antiviral agent are kept or grown the control of temperature, oxygen, CO₂ and/or nutrient supply. The exact parameters of said conditions may vary for each cell type, each antiviral agent applied, each type of virus infection, the stage of infection etc. Typically, cell culturing may encompass the use of a suitable vessels with a substrate or medium that supplies the essential nutrients, growth factors, hormones, and gases (i.e. CO₂, O₂) in a controlled and controllable manner. Obtained information may, in certain embodiments, also be recorded, e.g. together with Raman spectra or other diagnostic data. Furthermore, the vessel in which cells are grown may further be situated in a place or location, e.g. an incubator, where a physio-chemical environment may be regulated and maintained, including the pH, osmotic pressure, or temperature.

In certain embodiments, cells in a cell culture, e.g. infected cells or any other type of cells to be tested in accordance with the present invention, are grown to a desired or optimal density, i.e. having the optimal number of cells per volume of culture medium. Subsequently, infected cells may be lysed to release the cell content, which includes also the virus, parts of a virus, viral components etc. Alternatively, a cell culture may be grown as long as it takes to finish the viral replication program in the cells, leading to a release of the viruses (virions) to the extracellular space and a concomitant destruction of the cellular remnants. Virus particles and/or sub-portions thereof may be separated from other components via filtration, centrifugation or other techniques as described herein. The separated, isolated and optionally purified viruses or groups of viruses may, certain embodiments, be applied to a chip or microfluidic device as provided herein.

The herein described methods using cell culturing techniques may, in certain embodiments, be used for the development of vaccines, the control of antiviral compounds or strategies, the elucidation of viral replication, or any other research purpose.

In another preferred embodiment of the present invention the cells or group of cells are introducing into the chip, e.g. being a part of a microfluidic system as described herein, and are further analysed in the chip. The cells may, for example, be injected into the chip via an appropriate inlet as a liquid suspension. Prior to the injection into the chip, the cell suspension may be diluted to adjust the cell amount to defined concentration. For example, a dilution to single cells per predefined volume may advantageously be used for Raman spectroscopy, starting, e.g., at a concentration of 100 000 cells per ml. In cases where viral DNA or RNA is to be analysed, concentrations in the range of 1 to 100 μg/μl are possible. Cells may be diluted in an appropriate buffer, e.g. a buffer which is eligible for live cell analysis by Raman spectroscopy. Typically, phosphate-buffered saline (PBS) is used since it is isotonic and non-toxic to most cells.

In further preferred embodiment, a solution containing cells may be applied to the chip, wherein the cells are floating in the solution. In one embodiment, the cells may be floating in the chip due to microfluidic activities. For example, pumps or capillary forces may be used to direct or transport cells to the correct position on the chip, e.g. wells within the chip. The cells may not be kept in the wells, but are arrested only for certain time interval in order to allow for the performance of a spectroscopic analysis as described herein. In another embodiment, the cells are allowed to settle down in the wells within the chip. In a preferred embodiment, the cells are allowed to settle down in μ-wells within the chip. The depth of the μ-wells may, for example, have a size range from 5 to 50 μm in diameter, in increments of 5 microns. The μ-wells preferably have the same depth as their diameter to avoid to swirl the cells out of the wells with generation of a fluidic stream. Further information on suitable μ-wells can be derived from suitable literature sources such as Sekhavati et al., 2015, Integr. Biol., 7, 178.

It is herein envisaged to also determine viral infection pathways, infection progression or the duration of an infection, or to determine the effect of an antiviral treatment by Raman spectroscopy. It is thus also envisaged by the present invention to start virus infection and/or antiviral treatment after placing the cells onto the chip. In one embodiment, the deliberate infection of a cell with a virus or the antiviral treatment is performed subsequent to the introduction of the cell or group of cells into the chip and/or after a control measurement. In yet another embodiment, the deliberate infection of cells with a virus or the antiviral treatment is performed subsequent to the settling down of the cells into said wells and/or after a control measurement. It is further particular preferred to use collect and/or arrest cells on the chip via optical trap approaches and/or a focused laser microbeam as described herein.

In another aspect, the invention further relates to an in vitro method for analysing whole blood samples or samples comprising cellular portions of blood as to the presence of a virus infection of a cellular portion of blood, preferably of erythrocytes present in the sample comprising: a) spectroscopically analyzing said samples for the status of hemoglobin or of constituents thereof by means of spontaneous Raman spectroscopy; and b) comparing the spectroscopic data to a database and detecting the virus infection.

The term “whole blood” as used in the context of the in vitro method refers to a blood sample having all its components intact that has been withdrawn from a donor into an anticoagulant solution. Whole blood samples comprise thus erythrocytes, leukocytes, thrombocytes and blood plasma, i.e. a composition comprising dissolved proteins such as serum albumins, globulins, fibrinogen; glucose; clotting factors; electrolytes; hormones; carbon dioxide; and oxygen. The term “cellular portions of blood” as used herein relates to cells or cell types present in a mammalian blood sample, preferably in a human blood sample. These cells or cell types include leukocytes, thrombocytes and erythrocytes. The leukocytes may be present as neutrophils, eosinophils, basophils, lymphocytes or monocytes, which are typically part of the immune system and function in immune response. Erythrocytes or red blood cells lack a nucleus and organelles and are typically marked by glycoproteins that define the different blood types. The cytoplasm of erythrocytes is typically rich in hemoglobin, which binds oxygen. Hemoglobin is an iron-containing oxygen-transport metalloprotein (pigment) in erythrocytes. It consists of a globin composed of four subunits each of which is linked to a heme molecule, that functions in oxygen transport to the tissues after conversion to oxygenated form in the gills or lungs, and that assists in carbon dioxide transport back to the gills or lungs after surrender of its oxygen. The heme group consists of an iron (Fe) held in a heterocyclic ring, known as porphyrin. It is particularly preferred to perform the analysis with hemoglobin containing cells, more preferably with erythrocytes or precursors thereof. It is further particularly preferred to perform the analysis with porphyrin containing cells, e.g. erythrocytes or precursors thereof.

The present invention envisages the analysis of blood samples for the status of hemoglobin or constituents thereof. The “status of hemoglobin or of constituents thereof” refers to a functional and/or conformational condition of the hemoglobin. The functional condition relates to the hemoglobin's property to become saturated (oxyhemoglobin) or desaturated (deoxyhemoglobin) with oxygen molecules. This condition is assumed be related, inter alia, to the iron's oxidations state in hemoglobin, wherein the iron ion may be either in Fe′ or in the Fe′ state, wherein Fe′ cannot bind oxygen. The status may further be related to or depend on the folding state of hemoglobin. Without wishing to be bound by theory, it is assumed that hemoglobin can change its form to methemoglobin, wherein said methemoglobin is largely unable to bind oxygen. It is further assumed that binding processes in the context of the hem component of hemoglobin, in particular protein portions being in contact with or associated to the porphyrin ring component, e.g. the 1-beta chain of hemoglobin, may contribute to a change from hemoglobin to methemoglobin or a conversion of hemoglobin to a non-functional conformation, i.e. a conformation which is no longer capable of transporting oxygen. A “modified constituent” as used herein may thus be sub-portion of hemoglobin, e.g. the 1-beta chain, or a portion which includes the hem or the porphyrin ring structure, or which is directly or indirectly associated with it and/or which has a functional influence on said hem section. The “conformational condition” hence means a structural modification of the hemoglobin. Such a modified version, may, for example be methemoglobin or any similar version thereof. This modified version may further show specific and distinguishable Raman spectra when analysed with methods according to the present invention, when compared to situations in which unaltered hemoglobin is present, e.g. when comparing Raman spectra of cells comprising normal or unaltered hemoglobin and of cells comprising structurally modified versions of hemoglobin. Without wishing to be bound by theory, it is further assumed that binding processes which lead to functional and/or conformational modifications of the hemoglobin may be caused or affected by viral components such as, for example, surface glycoproteins, in particular E2 glycoprotein, or envelope proteins, nucleocapsid phosphoproteins, or a non-structural virus protein such as the ORF7a or ORF8 protein, or homologues of any of the above. It is, in particular, assumed that such proteins derived from a coronavirus, such as SARSCoV-2, may cause the mentioned modifications to hemoglobin. The present invention thus envisages an analysis and detection of said modifications via the status of hemoglobin in order to determine whether a virus interaction has occurred. In certain embodiments, the modification of hemoglobin may be due to a change of porphyrin due to interaction with a virus protein.

The present invention thus encompassed methods, which are directed to the analysis of whole blood or blood cells, in particular erythrocytes, via Raman spectroscopy in order to determine the status of hemoglobin or of its constituents. The presence of modified hemoglobin or modified constituents thereof, e.g. of modified hem comprising components, may be seen as being indicative for a virus infection of the subject the sample is derived from or the of the effect of a virus infection on a cell or group of cells. In a particularly preferred embodiment, the presence of modified hemoglobin or modified constituents thereof, e.g. of modified hem comprising components, may be seen as being indicative for a Coronavirus, more preferably of a SARS-CoV-2 virus infection of the subject the sample is derived from, or of the effect of a Coronavirus, more preferably of a SARS-CoV-2 virus infection on a cell or group of cells.

For comparing the information, specific Raman spectra of hemoglobin comprising cells, preferably of erythrocytes or precursors thereof, different states or forms of erythrocytes may be analysed. For example, the analysis may including measurement of hemoglobin comprising cells from healthy subjects, and/or from subjects suffering from a viral infection, which may have been obtained at any point of time in the past. Raman spectra may further be derived from databases or previously recorded data sets. The databases may additionally be compared to or supplemented with information from previously recorded spectra or database information. Moreover, the database my further comprise reference spectra, i.e. Raman spectra obtained from a hemoglobin of known status or from a virus able to infect erythrocytes of known identity.

According to the present invention, the modification of the hemoglobin, assumingly by binding of a viral surface glycoprotein to porphyrin may be detected by the method as described herein. By comparing Raman spectra obtained from the blood samples of a subject potentially infected with a coronavirus with Raman spectra obtained from reference spectra or databased spectra from blood samples positive for coronavirus, this could identify a coronavirus infection.

In a further particularly preferred embodiment, the methods as described herein, e.g. the determination of viral infection or of exosomes and analysis of liquid samples with respect to the presence, identity and properties of a virus or exosome, or the method for monitoring a viral infection, or the method for monitoring the antiviral treatment effect in a virus infected/affected cell or group of cells, or the method for analysing whole blood samples or samples comprising cellular portions of blood as to the presence of a virus infection are performed in an automated or semi-automated manner. To be capable to determine viral infections in cells and/or virus or exosome properties and/or hemoglobin modifications or antiviral treatment effects automatically or semi-automatically, method steps as mentioned herein above may be performed in a computer-based manner. For instance, once viruses or virus infected/affected cells or any cell to be analysed, e.g. an erythrocyte, enter a detection, e.g. of a microfluidic system as described above, images may be acquired. By using suitable image analysis software and/or particle/cell tracking or particle/cell counting devices and/or software, specific cells or groups of viruses or exosomes may be recognized, highlighted and/or be virtually labelled. The corresponding activities may be performed automatically, or, in certain embodiments semi-automatically, e.g. by requiring a human interaction or by asking for confirmation by the operator. Upon completion of these steps, additional analysis steps may automatically be started such as performance of stimulation of the cells, viruses or exosomes, spectral, e.g. Raman analyses, recording of spectra, e.g. Raman spectra, recording of bright field images of cells or viruses or exosomes, fluorescence of cells or viruses or exosomes, classification of viruses, quality control checks, comparison steps with visual images etc. Correspondingly obtained information may further be accumulated, stored in suitable databases or on suitable servers, transferred to remote systems or entities etc. It is preferred that all images taken are saved on a local hard disk and/or on a cloud server, at least until a sample or group of viruses, cells or exosomes has entirely been analysed. The saving time may further be extended for documentation purposes.

In further embodiments, the automatic determination or analysis may comprise a scanning activity, wherein preferably a predefined number of Raman spectra are collected automatically in a defined area. It is thus preferred that the concentration of cells or viruses or exosomes is set or kept at a suitable, typically high value so that with switching on the laser one cell, virus or exosome is caught, the Raman spectrum is taken. Subsequently, the laser may be switched off and the system may move to a different position, e.g. in a predefined distance, where the steps are repeated, i.e. the laser is switched on, a new sample, e.g. cell, virus or group of viruses, or exosome, or group of exosomes is arrested, then measured and released etc. The defined area may, for example, be a sub-portion of the zone where the cells, viruses or exosomes are located. By scanning a defined area, it is possible to determine how many cells, viruses or exosomes are present within the area. The scanning approach may be connected with the addition of a virtual label to each cell, virus or exosome, i.e. a tracking activity. The scanning may include the performance of spectral analyses as defined herein, e.g. Raman spectroscopy as mentioned above.

In a particularly preferred embodiment, the spectral analysis is performed by suitable and unique data analysis software, e.g. CT-RamSES, which is capable of processing and analysing Raman spectra taken from biological samples. It is preferred that the data analysis software provides fast spectral processing, safe data storage and easy statistical data analysis for biomedical data interpretation. For example, spectral data are imported from a control software and are subsequently automatically processed by the data analysis software. The software accordingly provides the data analysis plots. The underlying process includes organizing raw spectra of different data sets after conducting all spectral processing steps of (i) Smoothing (noise and cosmic spike removal) (ii) baseline corrections (intrinsic glass-background scattering removal) (iii) vector normalization (laser and instrumental effects removal, standardizing all spectra). Subsequently, mean Raman spectra with standard deviations can be calculated for each data set separately. Subsequently, principal components analysis may be conducted on the processed data sets, resulting in score plots describing the similarity and differences between the analysed data sets in form of a scatter plot. In a further embodiment, loadings of principal components may be presented in many plot forms: loadings peaks, bar, and histogram, indicating the spectral variations between the data sets that have been used in the analysis. These spectral variations are assigned to its respective biochemical changes. In a further embodiment, cluster analysis using K-means is designed and used to classify all measured spectra into groups of similar patterns, which can be used to identify diversities and subclasses within one measured heterogeneous sample.

In a further aspect, the present invention relates to a device for analysing a liquid sample as to the presence, identity and properties of viruses, wherein the device comprises as a first unit (i) a chip, optionally comprising a filtering unit, as a second unit (ii) a Raman spectroscopy system; and as a third unit (iii) an evaluation module which is coupled to the Raman spectroscopy system.

It is preferred that the device comprises a chip as defined herein above in the context of the methods of the present invention. The chip may, in certain embodiments also, e.g. optionally, comprise a filtering unit, e.g. as defined herein above or providing functionalities as mentioned above. The filtering unit may, for example, be required in case samples with heterogenous content are present, e.g. if two or more differently sized particles or elements need to be separated. Details on suitable filters or pore sizes or combinations of filters, which are all envisaged herein, can be derived from the corresponding section on filters in the methods portion herein above. In preferred embodiments, the filtering unit of the chip is designed to size-exclude components within the liquid sample which are larger than viruses or exosomes. Thereby viruses and/or exosomes may be isolated or enriched.

The second unit of the device, i.e. the Raman spectroscopy system, may comprise a light source which can in particular be a laser. The light source is configured to output an excitation beam. The excitation beam can for example have a wavelength in the range between 532 nm and 1064 nm, e.g. approximately 785 nm. A Raman spectrometer receives light scattered on the sample, e.g. a cell as defined above, by Stokes processes and/or Anti-Stokes processes. The Raman spectrometer can comprise a diffractive element and an image sensor in order to record the Raman spectrum of the sample. The Raman spectroscopy system can comprise further elements in a manner known per se, for example focussing optical elements which can be designed as lenses, and/or diaphragms.

The third unit of the device, i.e. the evaluation module, can be a computer or can comprise a computer. The evaluation module may be coupled to the Raman spectroscopy system and/or the microscope system as defined herein above. The evaluation module can control the recording of the Raman spectrum by the Raman spectroscopy system, as well as the visual and/or fluorescent recording of the viruses, cells or exosomes. In addition, the evaluation module comprises an interface in order to receive data from an image sensor of the Raman spectroscopy system or the microscope system. The evaluation module may comprise an integrated semi-conductor circuit which can comprise a processor or controller and which is configured to evaluate the recorded images or Raman spectra in order to determine the identity of a virus, of the virus-infection status of a cell. The integrated semi-conductor circuit is configured to determine by means of the Raman spectrum, optionally in combination with interpretation of visual images, the presence, identity and properties of a virus, virus infected/affected cell or an exosome. The integrated semi-conductor circuit as mentioned above can be configured to identify the presence or absence of determined Raman peaks or to determine the spectral weight of Raman peaks which relate to the identity of a virus, or the infection status of a cell, e.g. an erythrocyte.

In a further preferred embodiment the evaluation module is designed to perform principle component analysis as defined herein above. Additionally or alternatively, it may be designed to perform a normalization on a specific band and/or a cluster analysis as defined herein above. It is further envisaged that it may additionally or alternatively perform a hierarchical cluster analysis and/or a LDA analysis as defined herein above and/or supervised cluster analysis and/or deep learning.

In a further preferred embodiment, the evaluation module is designed to perform a statistical evaluation and judgment on the basis of artificial intelligence and/or machine learning algorithms for complex matrix data evaluation. A corresponding evaluation makes use of methods for artificial intelligence and/or machine learning algorithms for complex matrix data evaluation as described herein above. It is preferred that training data are obtained from previous, e.g. supervised, analyses and/or are derivable from databases as described herein.

In yet another preferred embodiment, the evaluation module is configured to analyse an isolated cell or virus by comparing the Raman spectrum obtained from an isolated cell or virus with a reference spectrum, preferably derived from a database. Databases and reference spectra correspond to those mentioned herein above in the context of the methods of the present invention.

The evaluation module can also comprise an optical and/or acoustic output unit, via which the information dependent on the analysis of the Raman spectrum is output, which shows, for example, whether or not antiviral effects on a cell have been identified. The output unit can also be structurally integrated into a housing of the evaluation module or of the Raman spectroscopy system.

The evaluation module can further comprise a memory in which comparative data is stored which the integrated semi-conductor circuit can use when evaluating the Raman spectrum. Information regarding the position and/or the spectral weight of different Raman peaks for analysed viruses, cells or exosomes can be stored in a non-volatile manner in the memory of the module. Alternatively or additionally, the information regarding the position and/or the spectral weight of different Raman peaks for the analysed cells, viruses or exosomes can be determined by the module by means of methods of supervised learning or other machine learning techniques.

In a further embodiment, the device according to the present invention additionally comprises as a fourth unit a microfluidic component or system, e.g. for semi-automated measurements of viruses and/or for transporting viruses, cells, groups of virus or cells, or antiviral agents and/or for separating said liquid sample components or viruses or cells, which is coupled to the Raman spectroscopy system.

The microfluidic component or system may essentially comprise the elements and components as described above in the context of the microfluidic system mentioned in the methods of the present invention. The microfluidic component may, for example, be configured to allow semi-automated or automated measurement of viruses, virus infected/affected cells or exosomes. It may in addition or alternatively be configured to transport a liquid sample, culture medium, waste, size-excluded particles, fragments, cell debris etc. It may further or alternatively be coupled to the evaluation module as defined herein above and/or the control checkpoint, which typically resides downstream of the filtration unit, in the chip as described above. Briefly, it may allow for a precise control and manipulation of fluids. It may further comprise active elements such as micro-pumps or micro-valves. It may further comprise a reservoir for cells, viruses or exosomes and a reservoir for fluids or buffers etc. It may additionally enable the isolation and collection of a virus, exosome or cell of interest, e.g. for further analysis, or cultivation or breeding, e.g. for further examination in the future or with an increased number of elements, e.g. further cells, viruses or exosomes. Envisaged analysis options include, for example, PCR analysis, analysis on DNA microarrays, or sequencing analysis, e.g. via next generation sequencing or nanopore sequencing. In a further preferred embodiment, the device according to the present invention comprises an integrated optical trapping module. The optical trapping module is able to produce an optical trap for collecting and arresting viruses, cells or exosomes therein, in order to record a Raman spectrum. The optical trap can be produced by the excitation beam of the Raw man spectroscopy system or a beam of electromagnetic radiation different therefrom.

The excitation beam can thus be used both as excitation for the Raman scattering and for producing the optical trap. Alternatively, the optical trap can also be produced by a separate beam. The Raman spectroscopy system can also comprise a light conductor, for example an optical fibre, by means of which the excitation beam and/or the Raman scattered light is guided. The light conductor can be positioned such that the excitation beam leaving said light conductor produces the optical trap with a focal point. In further specific embodiments, the optical trap may be split into several beams to simultaneously trap a multiple number of viruses, cells or exosomes.

In a further embodiment, the device may additionally comprise a module allowing for cell culturing. This module may be an integral part of the device or may be connected to it (and separable therefrom) by tubes or other connections. In certain embodiments, the module my be part of, integrated into or connected to a microfluidic system, e.g. as defined herein above. The cell culturing module may, in preferred embodiments, be designed for growing cells, preferably animal or mammalian cells, more preferably human cells, under controlled conditions outside their natural environment. The cells may be provided in a monolayer form, i.e. adherent to a substrate, or they may be freely floating, e.g. in a suspension like form. These conditions may vary for each cell type. It may provide reservoirs or inlets for the influx of culturing medium comprising, inter alia, essential nutrients such as amino acids, carbohydrates, vitamins or minerals, as well as growth factors, hormones. It may further comprise outlets for liquids or waste. It may further comprise inlets and outlets for gases such as CO₂ and/or O₂. In further preferred embodiments, the module allows for controlling the growth and/or natural status of a cell o group of cells. The module may, for example, comprises control units to measure and optionally change or adapt physio-chemical environment parameters such as pH, osmotic pressure and temperature. It may further comprise control units for measuring the concentration or amount of nutrients or essential compounds required for growth, e.g. amino acids, vitamins etc. These control units may, in certain embodiments, also be connected to effector units allowing for a change of concentration of the mentioned compounds, e.g. by opening an inlet from a corresponding reservoir or the like. The unit may further comprise a sector, zone or inlet designed for the introduction of virus particles to allow for a deliberate virus infection of cells. Alternatively or additionally, the unit may comprise a module, e.g. reservoir or inlet, for administering antiviral agents, preferably as defined herein above. This module may further comprise a control unit, which allows for the measurement of the concentration of an antiviral agent.

In a further specific embodiment the present invention relates to a device for analysing a liquid sample as to the presence, identity and properties of virus infected/affected cells wherein the device comprises as a first unit (i) a chip, optionally comprising a μ well unit where individual cells could settle down, as a second unit (ii) a Raman spectroscopy system; and as a third unit (iii) an evaluation module which is coupled to the Raman spectroscopy system. The elements described correspond to those defined herein above.

In further aspect the present invention relates to a device for analyzing exosomes in a liquid sample. This device comprises, in certain embodiments, an exosome isolation unit. This unit may, for example, comprise a chip, preferably with a filtering or immunocapturing functionality. This chip may, for example, be capable of isolating exosomes from a liquid sample. It is particularly preferred that the chip is designed to size exclude components within the liquid sample which are larger than exosomes, thereby isolating said exosomes. It is further particularly preferred that the immunocapturing unit of the chip as described above is designed to capture and thus isolate exosomes via immunoaffinitive interactions between receptors on the surface of exosomes and ligands on the surface of the chip.

Alternatively, the device may comprise or be connected to an exosome isolation unit which is based on microfluidics as described herein above. This unit may be capable of performing isolating exosomes via acoustophoresis, electrophoresis-driven filtration, dielectrophoresis, magnetophoresis, on-chip centrifugation, inertial lift force, viscoelastic flow, microfluidic filtration and microfluidic immunoaffinity as described above. The device further comprises a unit which comprises a Raman spectroscopy system. This system may, in certain embodiments, be combined with integrated simultaneous trapping features. The system is generally designed to record a Raman spectrum of exosomes derived from or in a liquid sample. The device further comprises an evaluation module which may be combined with the Raman spectroscopy system via remote association or may be an integral part of the system.

In a further embodiment, the device may additionally be linked to a microfluidic component. This component is designed for semi-automated measurement of exosomes and/or transporting exosomes, cells, groups of exosomes or cells, and/or separating said liquid sample components or exosomes which is coupled to the Raman spectroscopy system. The microfluidic system may be identical to or be integrated with the microfluidic functionality described above in the context of exosome isolation.

In preferred embodiment, the device further comprises an integrated optical trapping module or is designed as integrated Raman trapping microscope-spectroscope system. It is further particularly preferred that the device is configured to identify a Raman spectrum of the exosomes associated with a disease of the subject, in particular a disease as defined herein above such as cancer, a neurodegenerative disease, diabetes mellitus or a viral infection.

The device of the present invention is, in specific embodiments designed to perform any of the method according to the present invention as described herein.

A further aspect of the invention relates to a system comprising the device and a module comprising a database comprising reference values, e.g. from alternative sources such as molecular diagnostics, e.g. PCR or antibody based methods, obtained from a virus or a cell infected with a virus. In further embodiments, also reference values from Raman spectra are used. Said module refers to an integrated database of such reference values, e.g. PCR or diagnostic values or Raman spectra that were obtained, for example, from different setups or with controls, or based on known values, e.g. from previous measurements, or from previous or simultaneous control experiments, or from any literature source. The control experiments may comprise, for example, identifying viruses or virus infected/affected cells or exosomes with conventional methods known in the art, such as PCR, MALDI-TOF, and subjecting the identified cells or viruses or exosomes to Raman spectroscopy to record the respective Raman spectra. Said Raman spectra may then be fed into a database and used as comparative reference spectra for identifying viruses or exosomes from liquid samples, or for deciding whether a cell is infected with a virus, preferably for deciding with which virus a cell is infected, or whether a subject is affected by a disease.

Finally, the invention relates to a use of the method as described herein or the device as described herein or the system as described herein for the detection of a virus or a virus infection in a subject. Furthermore, the method, device or system may be used for determining antiviral effects on viruses or virus infected/affected cells. In further embodiments, the method, device or system may be used to determine dynamics of viral infections, e.g. in cell cultures. In a different set of embodiments, the method, device or system may be used for the analysis of blood samples as to the presence of virus infections, preferably in the context of erythrocytes or hemoglobin containing entities. In yet another aspect the present invention relates to the use of the method as described herein or the device as described herein or the system for the detection of a disease in a subject, preferably for the detection of cancer, a neurodegenerative disease, diabetes mellitus or a viral infection.

The figures and drawings provided herein are intended for illustrative purposes. It is thus understood that the figures and drawings are not to be construed as limiting. The skilled person in the art will clearly be able to envisage further modifications of the principles laid out herein.

### EXAMPLES

### Example 1

### Fast Identification of SARS-CoV-2 Infected Vero Cells

Raman trapping microscopy allows for fast detection and characterization of single cells, bacteria, exosomes or viruses and can also discriminate virus infected from non-infected control cells. Due to the implemented Trapping features individual cells are captured at the laser focus and hold tight during Raman analysis. The focused laser beam induces high photon density—creating a strong electromagnetic field gradient resulting in spectra of high intensity. This combination results in good and reliable spectra even samples that only differ in small portions from each other and has opened a new venue of applications especially for samples in solution in the sub-micrometer scale such as bacteria or exosomes and viruses.

Raman detection enhancement: Many approaches were developed to enhance Raman signals such as using Plasmon/resonance effects in Surface-Enhanced Raman Scattering (SERS), to enable Raman measurements of small cells. However, it requires chemical modification of the sample (applying nanostructures) and special coatings of the substrate surface. Thus, it is a sample destructive and time intense analysis.

In contrast, due to raise of spectral intensity (>>10 fold) by laser focusing BioRam® analysis cells, bacteria or viruses in minutes—direct within their native environment and in a highly automated manner.

The spectral changes of Vero cells infected with SARS-CoV-2 (COVID-19) was demonstrated at certain time points after infection.

Experimental setup: Virus infected cells as well as the corresponding control cells (the cell culture was aliquoted i.e. divided into several groups to allow control measurements of cells having identical culture conditions and culture time as the infected groups) have been fixed using 4% paraformaldehyde for 3 to 5 minutes and washed with buffer solution (PBS).

To monitor course of viral infection fixation was done at different time-points after incubation with virus: 0 hrs (control); 6 hrs and 24 hrs—after virus incubation.

The laser focus is around 1 μm in diameter which allows to measure distinct areas within a cell with subcellular spatial resolution. i.e. for measurements 1-10 flags were set per nucleus or within the surrounding cytoplasm. Measurements were automatically performed at the preselected sites.

For each sample, about 30 spectra are collected from cytoplasm and 30 spectra from nucleus of the Vero cells, which are injected in micro-channels of a chip with borosilicate glass bottom. Raman spectra were acquired using a 785 nm laser of 80 mW laser power for 5×3s, using a 60×water immersion objective, corrected to 0.17 mm.

BioRam usually collects data point at the spectral range 350-3000 cm⁻¹ with spectral resolution of 1 cm-1, meaning 2651 data points of 1 spectrum.

Data pre-processing: For statistics, data was cropped to 350-1800 cm⁻¹ as within this range most of the biological information can be found. This corresponds to about 1451 data points per each measurements. Then, the baseline was calculated by an asymmetric least squares fit, spikes were removed, and the spectra were smoothed with a median filter (window 3). Finally, the spectra were interpolated to continuous wave numbers and normalized using a Unit-Vector-Normalization.

Spectral bands assignments references: ACS. Talari, Applied Spectroscopy Reviews, 2015, 50:46-111. doi:10.1080/05704928.2014.923902.

Statistical Data Analysis: Principal Component Analysis (PCA) was used for visualizing the datasets. PCA was implemented in Python 2.7, using the scikit-learn package (Pedregosa et al., 2011, The Journal of Machine Learning Research, 12, 2825-2830) PCA Score plots were used to find clusters among the data and PCA Loadings enabled to find responsible wave number areas. Similarly, Linear discriminant analysis (LDA) were implemented using the sklearn Python library, which is applying linear transformation to the data followed by using PCA on the clustering mean. LDA transformations use the knowledge about the clusters from the training data to remove the within-cluster correlation, computed with a Singular Value Decomposition. This leads to a linear weighting of the columns, which is then used to decide the corresponding cluster of unknown data. Cluster analysis were applied using hierarchical cluster analysis (HCA), that define clusters based on the dissimilarity between the data points.

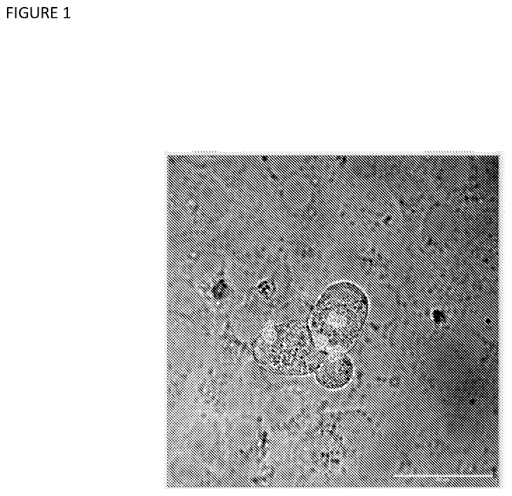

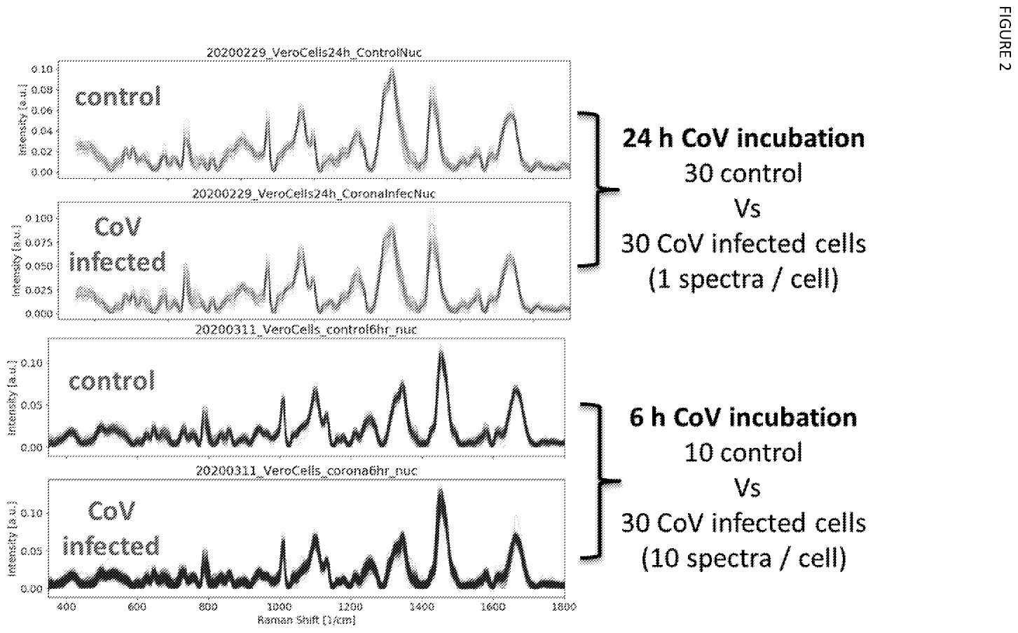

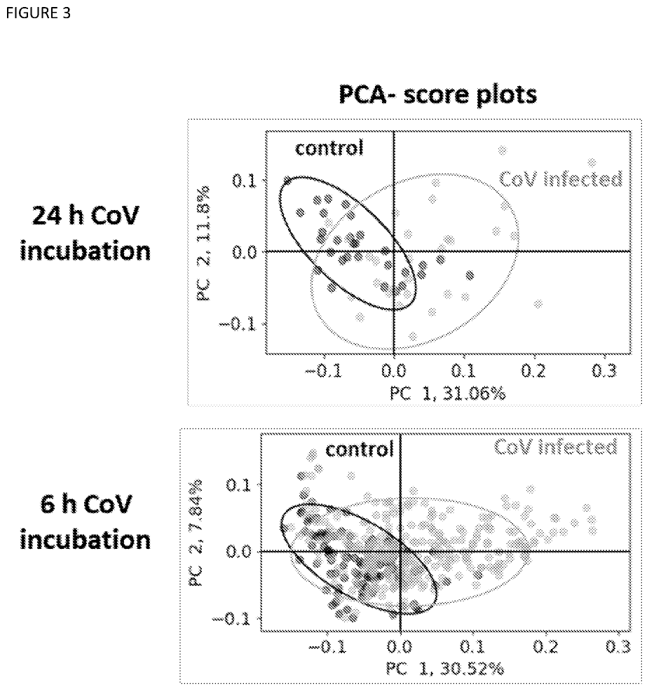

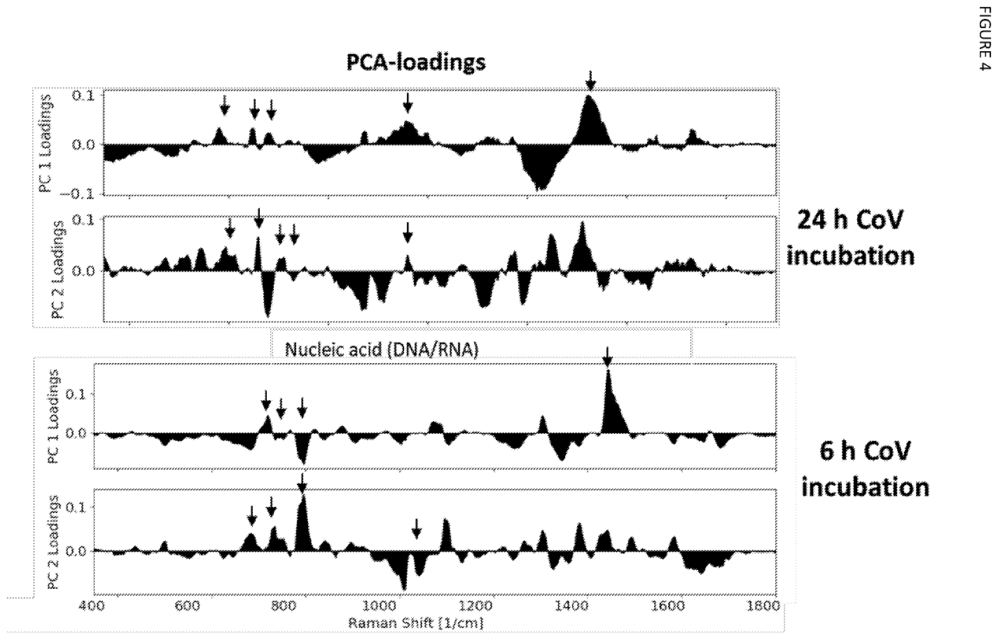

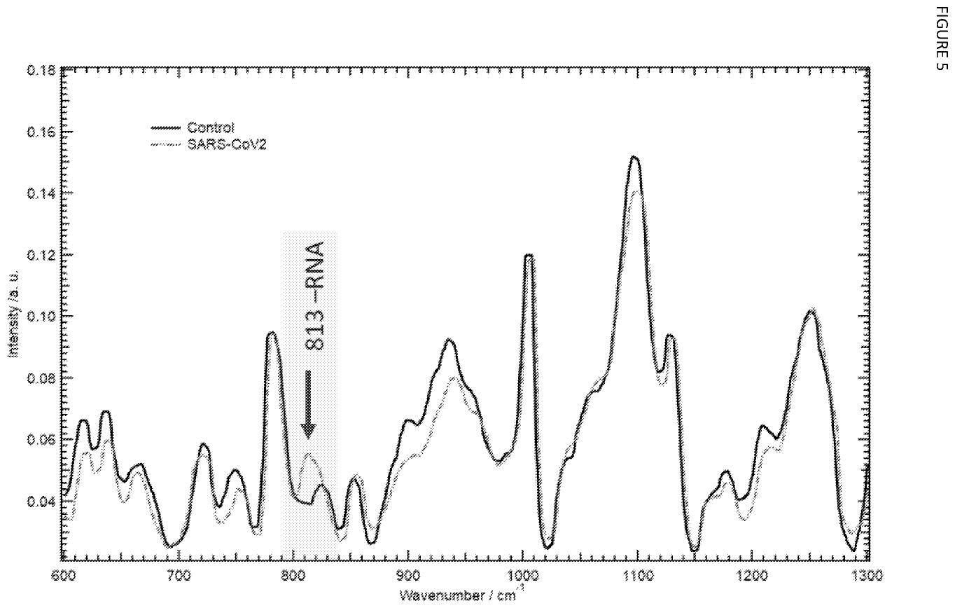

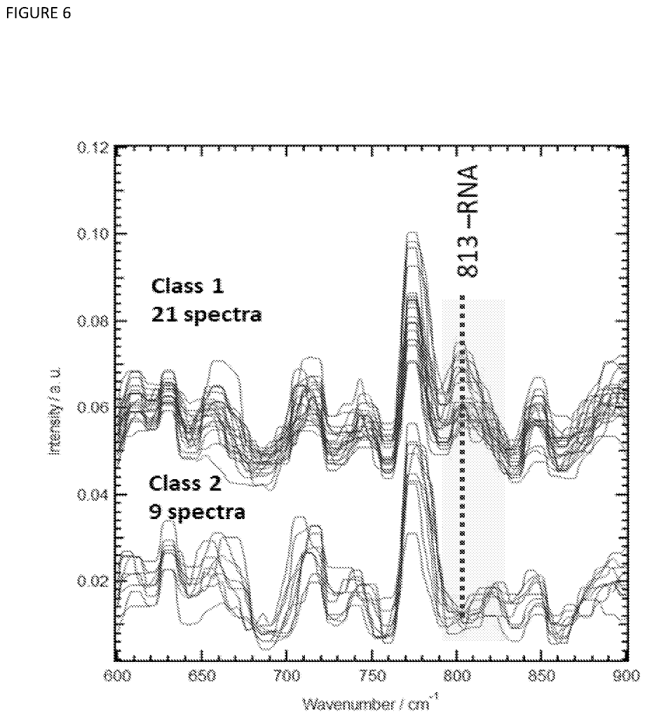

Results of the experiments can be seen in FIGS. 1 to 6.

### Example 2

### Identification of Influenza a Virus Infected A549 Human Lung Cancer Cells

A549 cells (also referred to as hA549 or A-549) are a specified, human cell line used in molecular biology and virology for cell cultures. They were derived from an explanted adenocarcinoma of the lungs of a 58-year-old white American. The cells were established by Donald J. Giard at MIT in 1972. A549 cells are typically used for pharmacological studies, transfection and virus propagation.

A549 cells are hypotriploid with different chromosome populations of around 66 and multiple mutations. They synthesize a relatively large amount of lecithin, using the cytidine diphosphocholine pathway. The cells grow adherently as a monolayer, RPMI1640, Ham's F12K or DMEM are used as cell culture medium, each with the addition of FKS.

Virus infected as well as control A549 cancer cells were injected into channels of a chip with borosilicate glass bottom. Measurements were taken from nucleus area-one spectrum per each cell. 100 cells from control sample were compared with 100 measurements of Influenza virus A (IVA) infected cells.

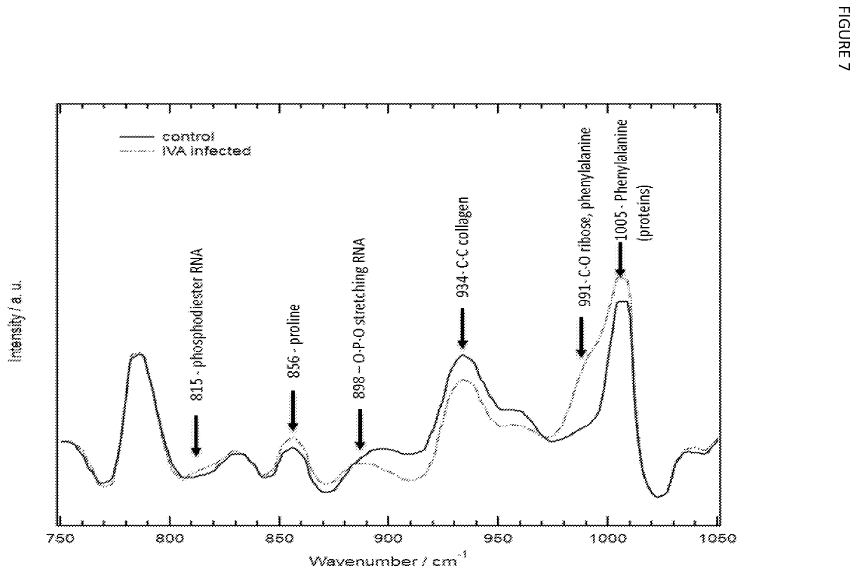

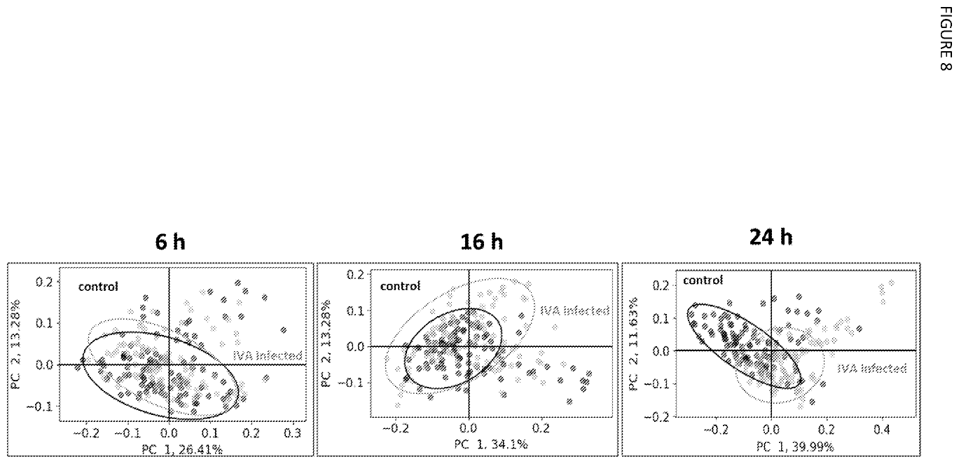

Results of the performed experiments are shown in FIGS. 7 and 8.

### Example 3

### Detecting the Virus within Supernatant of Virus Producing Cell Culture

Raman spectra were collected from influenza virus A (IVA) in solution via trapping.

Laser trapping was used to capture virus particles by moving the laser spot in solution for around 30 seconds, followed by Raman measurements using 785 m laser of 80 mW for 15 s (3s×5 accumulation). In alternative approaches the use of a laser of 300 mW reduces the measuring time to 1-4 sec.

5 spectra were collected from this sample.

Since the sample is extracted from the supernatant of cultured cells, there could be a possibility of contamination with bacteria or extracellular vesicles like exosomes.

Raman spectra collected from IVA were compared with typical E. coli bacteria and exosomes.

Raman spectra of IVA shows different Raman pattern than E. coli and exosomes, implying that the spectral band collected from the virus sample are basically from the IVA and not from bacteria or exosomes that can contaminate the sample.

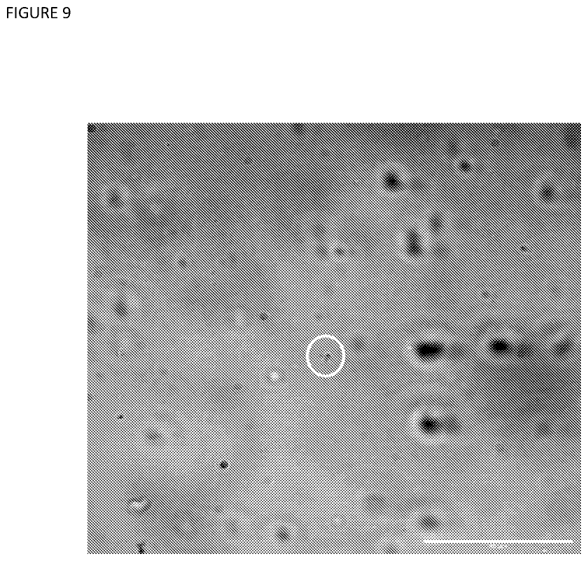

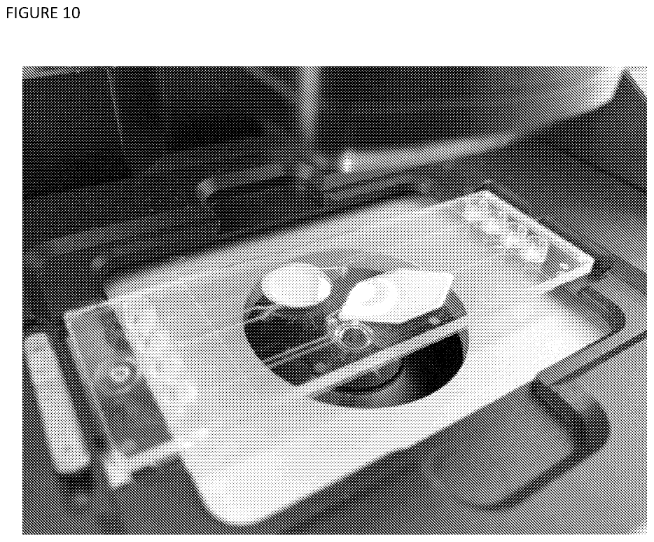

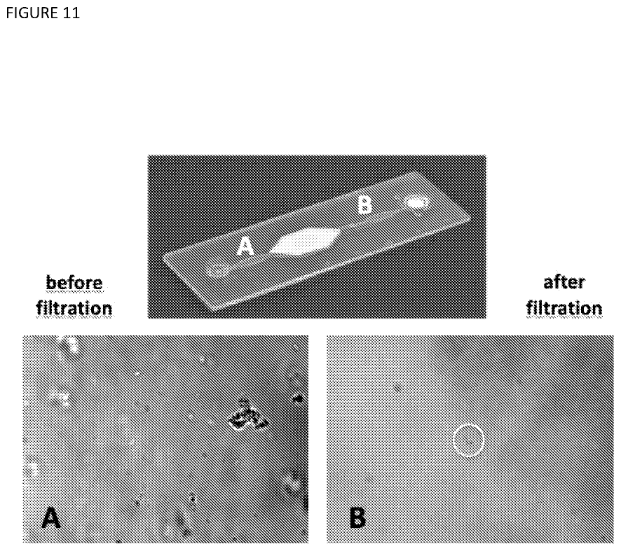

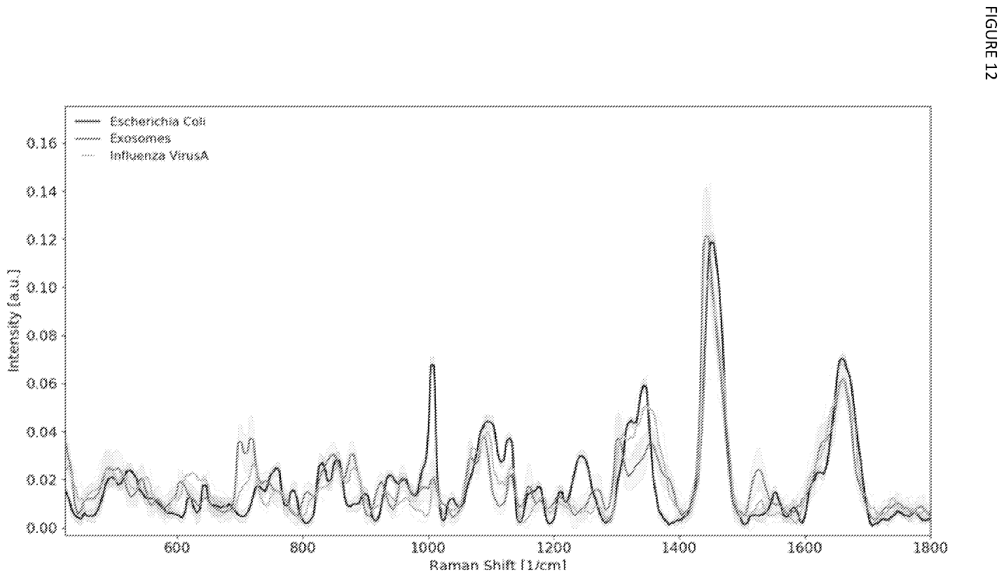

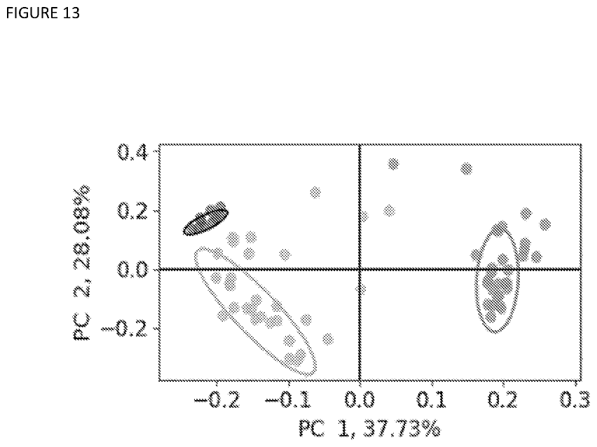

Results of the performed experiments are shown in FIGS. 9 to 13.

### Example 4

### Detecting of Oncolytic Viruses

Oncolytic Virotherapy. Targeted viruses are used for cancer therapy and companion diagnosis. Attenuated vaccine virus could result in dramatic regression and elimination of solid tumors in animals without damaging healthy tissues or organs. There are several products with broad applications for cancer detection and therapy. Patients treated with such viruses have to be free of virus before leaving the S2-hospital. The idea was to use Raman spectroscopy for testing.

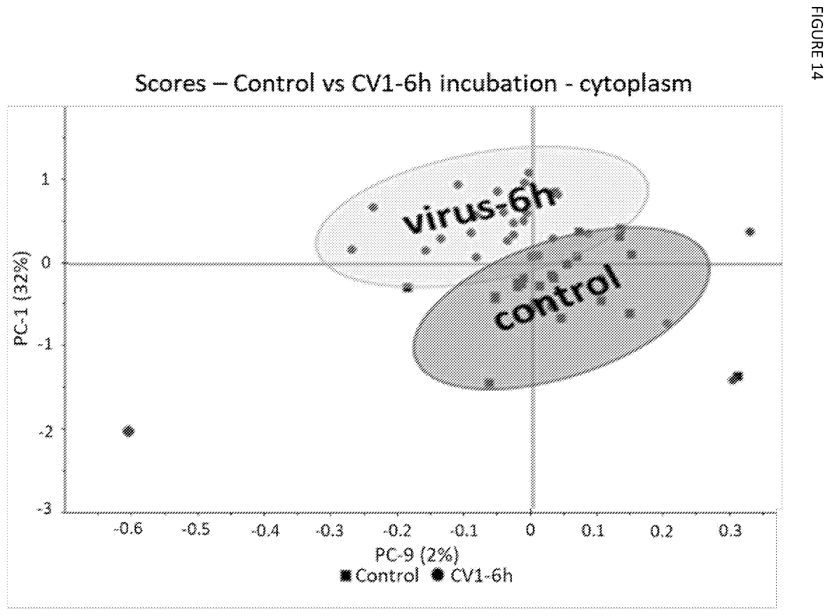

The results of this experiment are shown in FIG. 14.

### Example 5

### Raman Measurements of Blood from Healthy Donors and from COVID-19 Patients

It has been shown that SARS-CoV-2 attacks the 1-Beta Chain of hemoglobin and captures the Porphyrin which inhibits Human Heme Metabolism (Liu et al. 2020; doi.org/10.26434/chemrxiv.11938173.v8). Raman measurements of blood from 6 COVID-19 patients were performed and compared to results from 6 healthy donors. A droplet of whole blood was placed into a microchannel of a channel slide and measured. Interestingly the spectra from COVID-19 patients have similar results as compared to Raman data received from erythrocytes of blood products 40 days after donation. Results suggest that conformation changes of hemoglobin during aging seems to be comparable with those of COVID-19 patients.

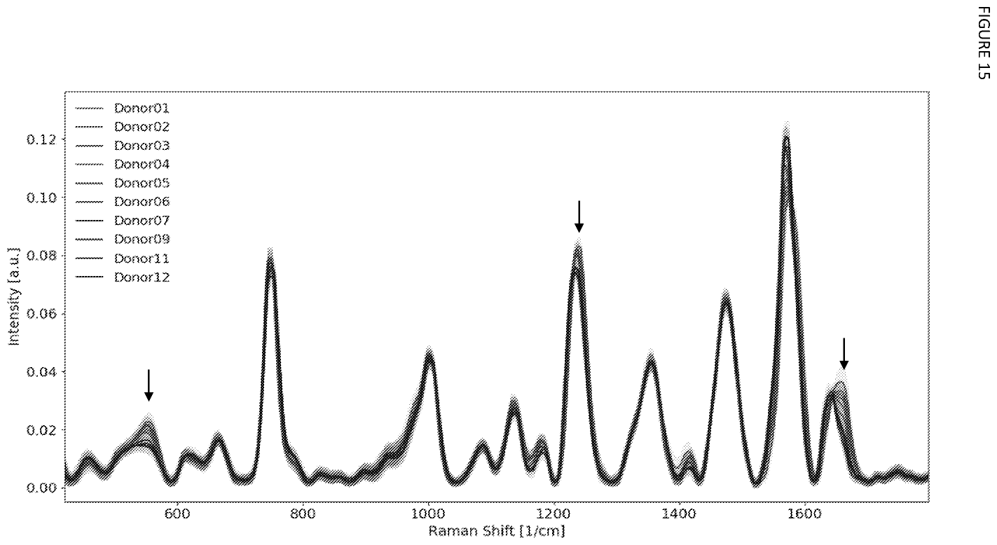

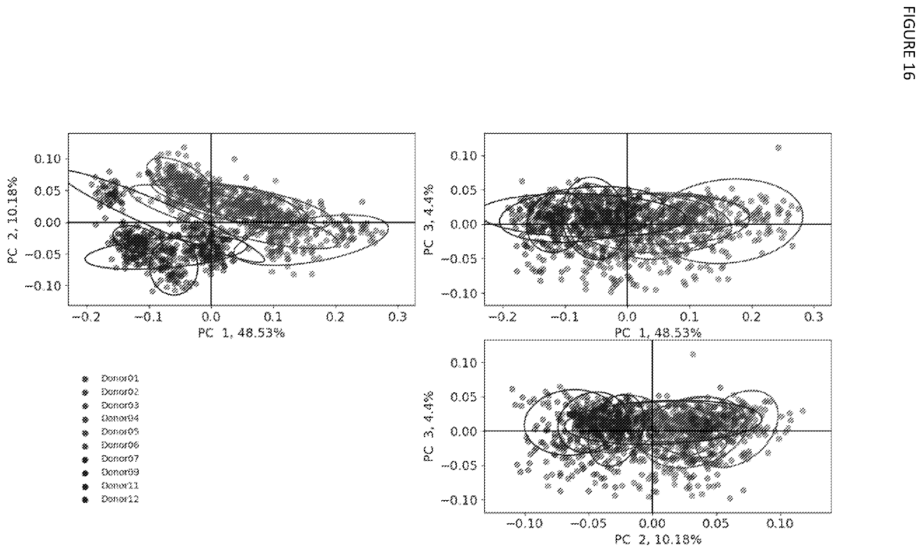

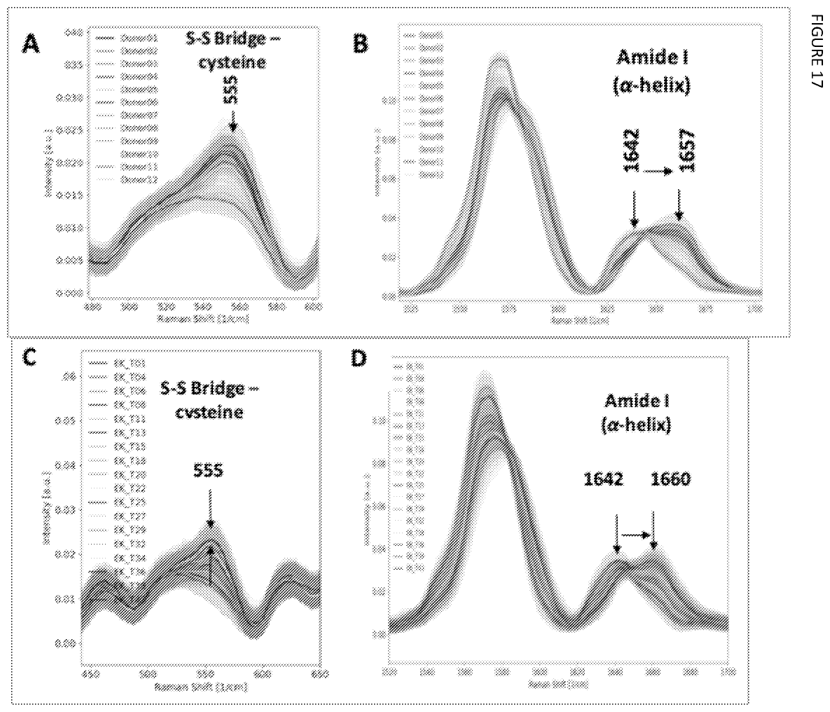

Results of these experiments are shown in FIGS. 15 to 17.

### Example 6

### BioRam® Analysis Workflow

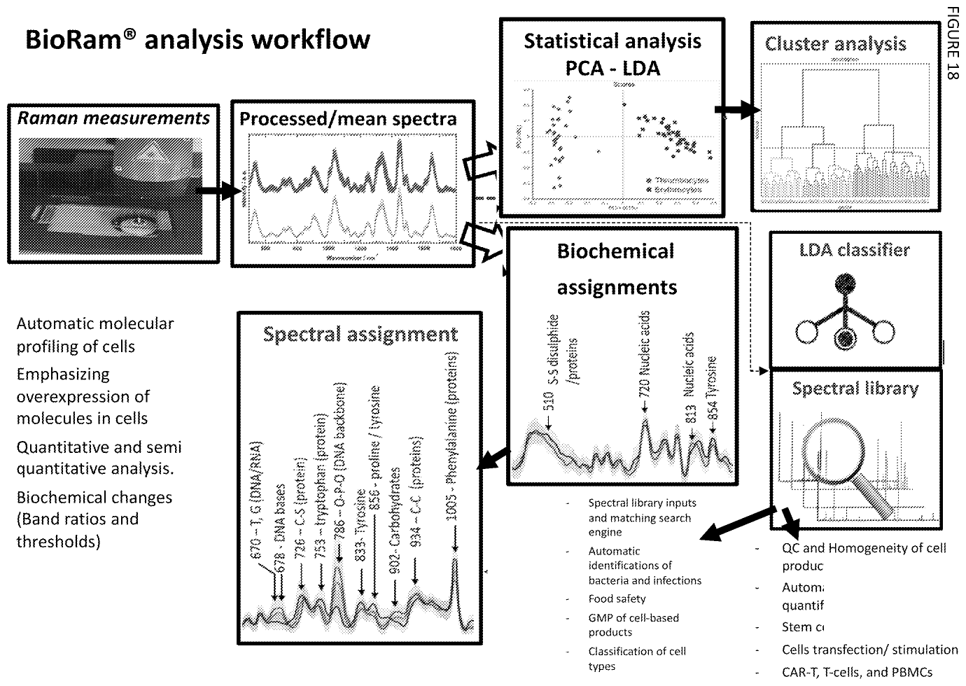

The BioRam® analysis workflow used for some embodiments of the present invention, as schematically shown in FIG. 18, comprises the following steps:

20 μl of the sample is pipetted into a microchannel chip and the chip is fixed on the stage of BioRam® microscope.

Raman measurements of cells are conducted using 785 nm/80 mw laser and accumulation time of 15 sec.

The measurements can be done during trapping of the cells or by selecting cells of interest and the measurements will conducted automatically.

The results are saved as raw Raman spectra with the photo of the respective analysed cell.

The raw spectra are extracted for data analysis.

The spectra are then baseline corrected, smoothed, cosmic spike corrected, and vector normalized.

The mean spectra is calculated from the processed spectra of each sample. The Raman bands in the mean spectra are assigned to specific vibrations of the respective molecule such as proteins and DNA.

After bands assignments, comparing the mean spectra of different samples can illuminate the differences in the biochemical composition of the samples and also follow biochemical changes over time.

The processed spectra may be analysed by different multivariate statistical methods:

1: Principal components analysis (PCA) is used to classify and compare different samples and can detect the spectral differences that can be used for classification.

2: Hierarchical cluster analysis (HCA) is use to classify the subclasses of mixed cells by detecting the dissimilarity between different types within one mixed population.

3: Linear discriminant analysis (LDA) is a linear transformation of the data that is applied after PCA to detect the small changes between samples and to train a classifier based on reference samples to build a classification model, then applying this model to detect cell types in a mixed population.

### Example 7

### Analysis of Exosomes

Samples:

Exosomes were Extracted from Patients Plasma:
Vascular disease patients (control group): P31, P32, P33, P34, 37
Colorectal cancer patients: P50, 52, 54, 57, 59.

Questions:

Can Raman spectra of exosomes and differentiate between the 2 categories?

Raman Measurement:

For each sample for 30 Raman measurements using laser trapping were collected. Raman spectra were acquired for 10×3s with a 785 nm laser and 80 mW laser power, using a 60× water objective, corrected to 0.17 mm.

Data Pre-Processing:

For statistics, data was cropped to 450-1800 cm⁻¹ as within this range most of the biological information can be found. Then, the baseline was calculated by an asymmetric least squares fit, spikes were removed and the spectra were smoothened with a median filter (window 3). Finally, the spectra were interpolated to continuous wave numbers and normalized using a Unit-Vector-Normalization.

Statistical Data Analysis

Principal Component Analysis (PCA) was used for visualizing the datasets. PCA was implemented in Python 2.7, using the scikit-learn package (Scikit-learn: Machine Learning in Python, Pedregosa et al., IMLR 12, pp. 2825-2830, 2011). PCA Score plots were used to find clusters among the data and PCA Loadings enabled to find responsible wave number areas.

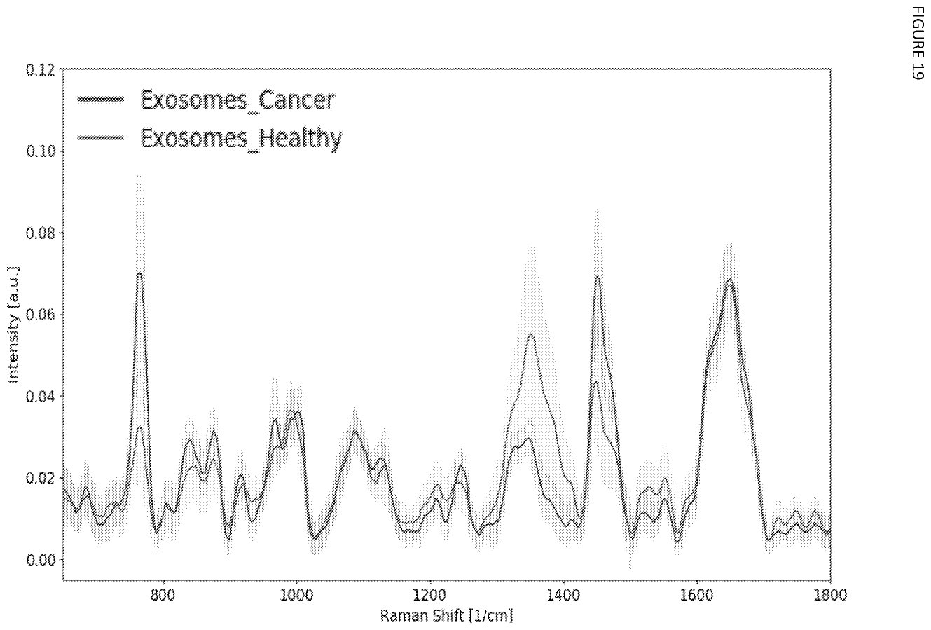

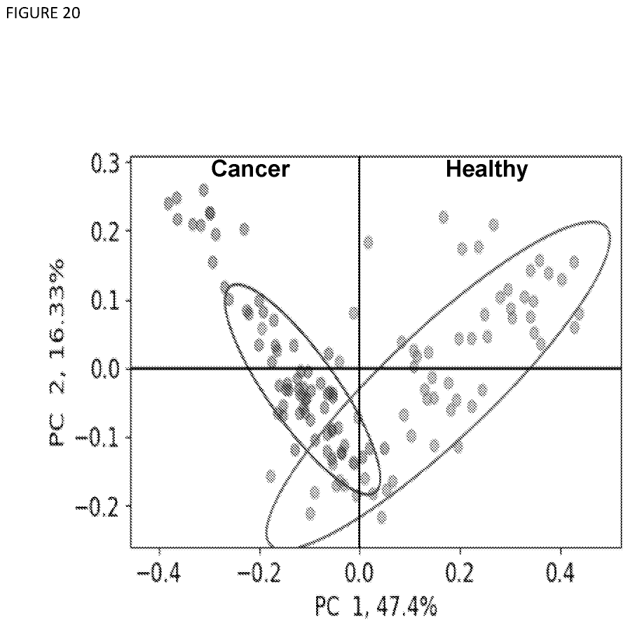

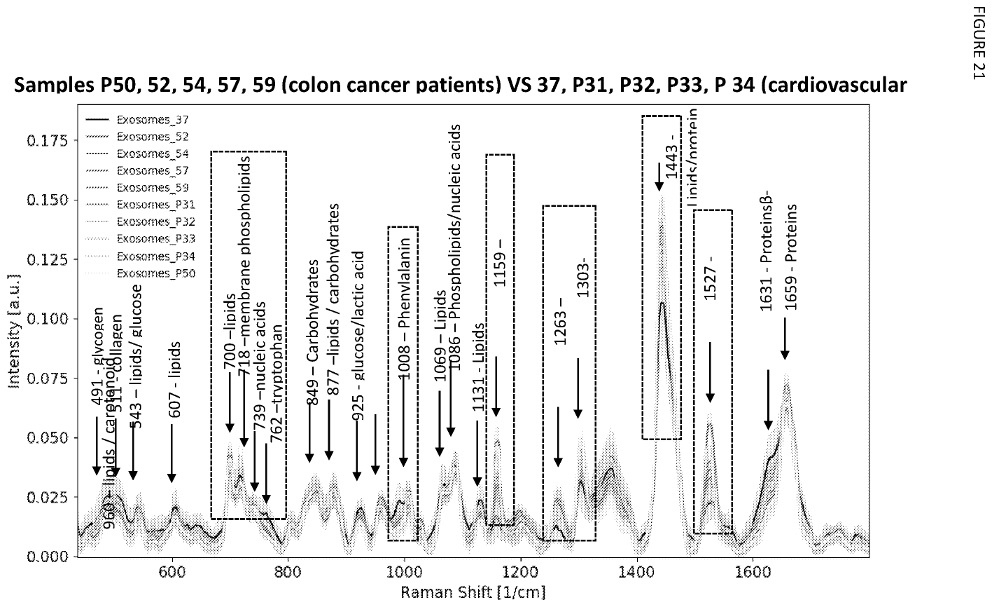

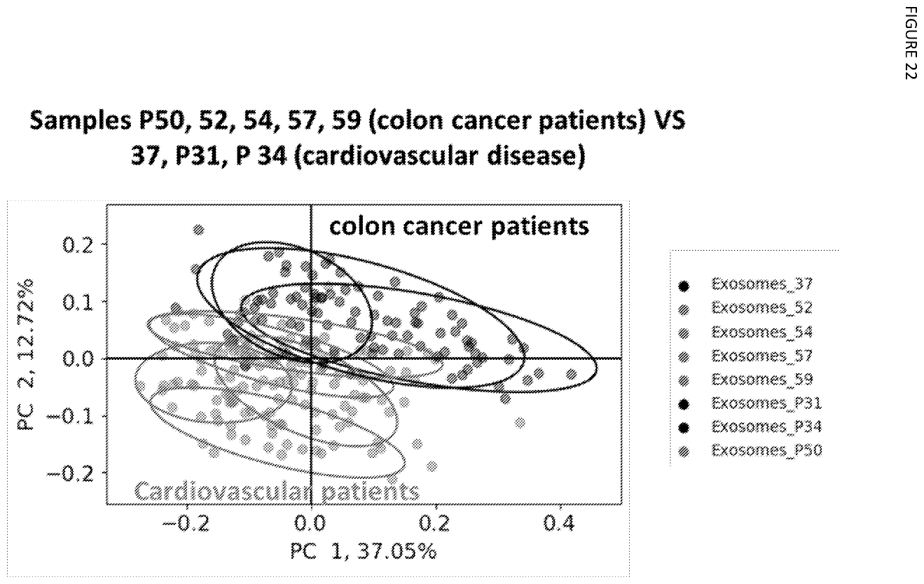

Results of these experiments are shown in FIGS. 19 to 22.

1. An in vitro method for analysing liquid samples as to the presence, identity and properties of a virus comprising:
a) analyzing said liquid samples for a virus spectroscopically by means of spontaneous Raman spectroscopy; and
b) comparing the spectroscopic data to a database and identifying said virus.

2. The method of claim 1, wherein said presence of a virus is a virus infection of a cell and/or indicates a virus infected cell.

3. (canceled)

4. The method of claim 1, wherein said analyzing step a) comprises an examination of cells and/or cellular compartments and/or cellular components such as extracellular vesicles, comprised in said sample.

5. The method of claim 4, wherein said examination comprises a separate examination of cellular compartments such as cell's cytoplasm and/or nucleus and/or nucleoli and/or mitochondria and/or lipid droplets.

6. The method of claim 4, wherein said viruses or cells are either unaltered or have been fixated.

7. The method of claim 1, additionally comprising as step a-(i) an isolation of the virus from the liquid sample.

8. The method of claim 7, wherein said step a-(i) is performed by cell lysis and sub-sequent centrifugation or filtration of said liquid sample, or by a centrifugation or filtration of said liquid sample or wherein the supernatant of a cell culture of infected cells is directly applied to a chip.

9. The method of claim 8, wherein said filtration is performed in a chip designed to size-exclude components within the liquid sample which are larger than the virus.

10. The method of claim 9, wherein the viruses are enriched in a micro-chamber of the chip, wherein said chip is preferably part of a microfluidic system.

11. (canceled)

12. The method of claim 1, wherein said step a) comprises recording at least one Raman spectrum by means of Raman spectroscopy of a virus.

13. The method of claim 12, wherein the analysis of step a) comprises collecting and arresting at least a group of viruses in an optical trap in order to record a Raman spectrum, preferably comprising collecting and arresting a group of free-floating viruses in an optical trap in order to record the Raman spectrum, or wherein the analysis of step a) comprises arresting a cell suspected to be virus infected or a cell derived from a cell culture of infected cells in an optical trap in order to record a Raman spectrum.

14. (canceled)

15. The method of claim 13, wherein said optical trapping forces are produced simultaneously by means of an excitation beam of a Raman spectroscopy system.

16. A method for monitoring a viral infection in a cell or group of cells, preferably in a cell or group of cells in a cell culture.

17.-18. (canceled)

19. The method of claim 16, wherein said cell or group of cells is derived from a cell culture or a patient's sample, and wherein said sample or cell culture derived cell or group of cells is or has been treated previous to or during the monitoring of the viral infection with an antiviral agent.

20. (canceled)

21. The method of claim 16, comprising recording at least one Ra-man spectrum by means of Raman spectroscopy of a virus in said cell or group of cells; or of a virus infected/affected cell or group of cells, preferably wherein said recording is performed previous and/or subsequent to the viral infection.

22.-72. (canceled)

73. A device for analysing a liquid sample as to the presence, identity and properties of viruses, wherein the device comprises as a first unit (i) a chip, optionally comprising a filtering unit, as a second unit (ii) a Raman spectroscopy system; and as a third unit (iii) an evaluation module which is combined with the Raman spectroscopy system.

74. The device of claim 73, wherein said device comprises a fourth unit (iv) a micro fluidic component for semi-automated measurements of viruses and/or for transporting viruses, cells, groups of viruses or cells, or antiviral agents and/or for separating said liquid sample components or viruses or cells, which is coupled to the Raman spectroscopy system, preferably further comprising a module allowing for cell culturing.

75.-77. (canceled)

78. The device of claim 73, wherein said filtering unit of the chip is designed to size-exclude components within the liquid sample which are larger than viruses, thereby isolating said viruses.

79.-98. (canceled)
